(19) United States
(12) Patent Application Publication
(10) Pub. No.: US 2026/0015568 A1
(43) Pub. Date: Jan. 15, 2026

(54) CELL PORATING AND OPTICALLY DETECTING MICROFLUIDIC DEVICES

(71) Applicant: Hewlett-Packard Development Company, L.P., Spring, TX (US)

(72) Inventors: Kenneth John Ward, Corvallis, OR (US); Viktor Shkolnikov, Palo Alto, CA (US)

(73) Assignee: Hewlett-Packard Development Company, L.P., Spring, TX (US)

(21) Appl. No.: 18/880,135

(22) PCT Filed: Jul. 15, 2022
(86) PCT No.: PCT/US2022/037280

§ 371 Date: Dec. 30, 2024

(51) Int. Cl. C12M 1/42; C12M 1/00; C12M 1/34; C12M 3/06

(52) U.S. Cl. CPC C12M 35/02; C12M 23/16; C12M 23/22; C12M 41/36

(57) ABSTRACT

Examples are directed to a microfluidic device that includes a reservoir to contain a fluid including at least a cell and at least a molecular probe, a cell-poration region fluidically coupled to the reservoir and including a cell-poration mechanism, and an optical-detection region fluidically coupled to the cell-poration region and including an optically transparent window associated with a wall of the optical-detection region. The microfluidic device further includes a fluid ejector fluidically coupled to the optical-detection region.

### BACKGROUND

Separation or sorting of cells may be beneficial for a variety of applications, such as in the field of biology and medicine. Cells may interact with molecules or compounds that bind to a target of the cell. Such species that bind to targets may be referred to as a marker and the targets may correspond to a specific cell phenotype or characteristic. Cell sorting may be carried out using fluorescence-activated cell sorting (FACS) devices, which may use focused sheathed flow in a quartz cuvette or other flow and deflect the fluid droplets containing cells based on the fluorescence response of the respective fluid droplet. Such systems are slow, may involve input of many cells, and are expensive to use due to the specialized fluidics and complicated electrical and optical components.

### BRIEF DESCRIPTION OF THE DRAWINGS

FIGS. 1A-1E illustrate example microfluidic devices with a cell-poration, optical-detection region, and fluid ejector, in accordance with the present disclosure.

FIGS. 2A-2B illustrate example cell-poration regions of a microfluidic device, in accordance with the present disclosure.

FIGS. 3A-3B illustrate example optical-detection regions of a microfluidic device, in accordance with the present disclosure.

FIGS. 4A-4C illustrate example microfluidic devices and coupled circuitry, in accordance with the present disclosure.

FIGS. 5A-5D illustrate different example apparatuses including a microfluidic device, optical sensing device, and circuitry, in accordance with the present disclosure.

FIGS. 6A-6D illustrate further example microfluidic devices with a cell-poration region, optical-detection region, and fluid ejector, in accordance with the present disclosure.

FIG. 7 illustrates an example method for porating and sorting cells using a microfluidic device, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Living cells are the basic building blocks of skin, tissues, and other biological materials. For example, Eukaryotic cells have a nuclease and other membrane-bound organelles enclosed within a cell membrane. The membrane-bound organelles enclosed within the cell membrane, such as the nucleus, mitochondria, and endoplasmic reticulum, may be associated with cell properties and functionalities that are of interest for testing, research, and other purposes. A gelatinous liquid fills the inside of the cell, referred to as the cytosol contained in the cytoplasm, which includes water, salts, and organic molecules. Some of the membrane-bound organelles may be enclosed by another membrane, such as nuclear membrane that encloses the nucleus, to separate such organelles from the cytoplasm. In a biological sample, a cell of interest or a target cell may be intermixed with other cells and components. The other cells and components may interfere with manipulation and/or analysis of the target cell. Isolating the target cell from other cells and components of the biological sample may allow for subsequent analysis or processing of the target cell without further interference. In some instances, target cells may be isolated by repetitively dispersing and re-aggregating samples, which may result in loss of the quantity of the target cell, loss of cell viability, and may be time consuming and labor intensive, thereby increasing time and cost for cell isolation. Further, many cell isolation techniques involve the use of surface markers, e.g., binding targets or regions on the surface of the cell. Surface markers may not access information contained within the cell and/or may limit the functionality used for cell sorting. Although the above describes Eukaryotic cells, examples are not so limited and may include Prokaryotic cells. Prokaryotic cells include a nucleoid region that contains genetic material (e.g., nucleic acid), ribosomes that make proteins, and cytosol that contains a cytoskeleton that organizes the cellular materials which are enclosed by the cell membrane. Prokaryotic cells lack a nucleus and other organelles, as well as internal membranes.

Examples of the present disclosure are directed to a microfluidic device which may sort live cells based on internal markers, herein generally referred to as intracellular targets. The intracellular targets are in the cytoplasm of the cell. To access the internal markers, the cells may be transformed or transfected via use of complexes of nucleic acid, proteins, protein fragments, and/or nanoparticles coupled with a detectable optical signal, such as fluorescent moieties, herein together referred to as a molecular probe that includes both the material to be introduced into the cell and the optical signal for detecting for the presence of the molecular probe. In some examples, the molecular probe includes a nucleic acid or a nucleic acid complex that encodes for a protein and the nucleic acid or nucleic acid complex is attached to a moiety, a molecule, or a compound exhibiting the optical signal. The microfluidic device includes a reservoir to store a fluid containing cells and at least a molecular probe, a cell-poration region having a cell-poration mechanism to transfect or transform the cells with the molecular probe, an optical-detection region with an optically transparent window to optically observe the resulting transfected or transformed cells, and a fluid ejector to selectively eject cells based on the optical observation. The porating, transfection or transformation, and optical detection all occur within the microfluidic device, which may reduce manual handling of the biological sample and/or reduce the volume of fluid used. The cells may be selectively ejected to isolated regions of a substrate based on the detected presence of the intracellular target. By sorting cells based on an intracellular target, the cells may be sorted by functionality rather than based upon just surface markers.

As an example, the intracellular target may be expressed due to a change in a signaling pathway. Many signaling pathways inside the cell do not produce a surface target, and thus use of intracellular targets may increase sorting ability and increase pathways which may be targeted for detection and/or further analysis or experimentation. As a specific example, a messenger ribonucleic acid (mRNA) may encode for a protein target, and the presence of the mRNA target may indicate activation of a signaling pathway. Additionally, cells which do or do not properly transfect or transform may be detected such that only transfected or transformed cells are isolated, and the cells are viable after sorting to allow for further analysis and/or culturing of cell(s). Transfected or transformed cells, as used herein, includes or refers to cells having the molecular probe introduced within the cell and bound to the intracellular target, such that the molecular probe is present inside the cell membrane.

As used herein, an intracellular target or marker refers to or includes a molecule and/or a set of molecules located or expressed by or inside a cell, e.g., is intracellularly expressed and located within the cell membrane which separates the cell from the extracellular environment. The molecular probe may be designed to bind to the intracellular target. In some examples, the intracellular target is expressed at a particular time and/or by a particular type of cell, such as when the cell is growing and/or in stem cells or differentiated cells. As the intracellular target may be expressed by particular types of cells and/or at particular times, it may be difficult to identify and assess such targets. Example intracellular targets include nucleic acid sequences, such as RNA or deoxyribonucleic acid (DNA) sequences, proteins such as enzymes, metabolites, or transcription factors, or lipids, among other molecules located within the cell membrane and/or in the cytoplasm of the cell. In some examples, the intracellular target includes an RNA sequence that encodes a protein. Intracellular targets may be associated with different signaling pathways, diseases or other conditions, among others. Particular intracellular targets may be assessed for diagnostics, risk assessment, therapy or drug development, research, among others. A target cell, sometimes referred to as “a cell of interest”, is a cell that expresses or includes the intracellular target.

Cell transfection is a process of introducing material, such as a molecular probe, within or inside a Eukaryotic cell. Cell transformation is a process of introducing material, such as the molecular probe, within or inside a Prokaryotic cell. To perform either cell transfection or cell transformation, apertures are formed in the cell membrane via a cell-poration mechanism and the molecular probe is allowed to pass or diffuse through an aperture to the inside of the cell, herein generally referred to as “cell poration” or “cell porating”. A molecular probe, as described above, includes a molecule (e.g., nucleic acid or a complex of nucleic acid, protein, protein fragment, nanoparticle) bound to a detectable optical signal, such as a moiety, molecule, or compound exhibiting the optical signal. Cells and their organelles are enveloped by thin membranes that separate their chemical contents from the environment outside the membranes. Cell porating may be performed by a cell-poration mechanism, such as those implementing electroporation and/or mechanical poration techniques. Electroporation, as used herein, refers to or includes forming apertures or pores in a cell membrane using electric fields. Mechanical portions refers to or includes forming apertures or pores in a cell membrane using mechanical forces.

For example, the cell-poration mechanism may include electrodes within a microfluidic channel used to perform electroporation and/or a constriction portion within the microfluidic channel used to perform mechanical poration. As the cell passes through the cell-poration region, the cell-poration mechanism is used to apply an electric field and/or shear force to or across the cell to cause formation of apertures in the cell membrane, which are sometimes referred to as “pores”. As used herein, an electric field refers to or includes a physical field or region surrounding a charged particle, e.g., the cell, which exerts forces on the charged particle by attraction or repulsion. A shear force refers to or includes a force caused by parallel forces acting on the cell in opposite directions and at different parts of the cell. The amount of electric field and/or shear force applied may be set to cause apertures of a sufficient size for a particular molecular probe to pass through. Too much electric field and/or shear force may result in cell lysis or death, and too little electric field and/or shear force may result in no apertures forming or apertures of an insufficient size for the molecular probe to pass through (e.g., for transfection or transformation and/or pores which are smaller than a diameter of the molecular probe). The electrical and/or mechanical poration may not produce contaminates, may be used to introduce the molecular probe within the cell (e.g., transfect or transform) without performing washing and at the single cell level, may be used to perform cell poration on multiple types of cells and/or introduce multiple molecular probes in one microfluidic device, and/or to optimize poration conditions for a particular type of cell and/or type of molecular probe. The molecular probe, as further described below, may output a particular fluorescent signal in response to binding to the intracellular target and the particular fluorescent signal may be detected within the microfluidic device.

The fluid ejector may be used to drive the flow of the fluid from a reservoir, through the cell-poration region, to the optical-detection region, and to selectively eject fluid from the microfluidic device based on observations made from the optical-detection region. The optical-detection region includes a wall or a portion of the wall that is optically transparent, herein generally referred to as an “optically transparent window.” In some examples, the optically transparent window forms a collimating lens or may include a collimating lens attached thereto. The optical-detection region may be partially defined by an opposing or transverse wall (relative to the optically transparent window) that includes or provides access to an optical detector. An opposing wall as used herein is a wall that is located across from the optically transparent window. A traverse wall is defined as a wall that is located along a plane that transects a plane of the optically transparent window, e.g., a perpendicular. Accordingly, an optical detector may be located with respect to the optically transparent window such that a light beam passes through the optically transparent window onto the optical detector or to an element capable of directing the light beam to the optical detector. In other examples, an optical sensing device separate from the microfluidic device may be used that includes a light source to provide excitation light toward the optically transparent window and an optical detector to measure emitted light responsive to excitation light.

Some examples are directed to a microfluidic device comprising a reservoir to contain a fluid including at least a cell and at least a molecular probe, a cell-poration region fluidically coupled to the reservoir and including a cell-poration mechanism, an optical-detection region fluidically coupled to the cell-poration region and including an optically transparent window associated with a wall of the optical-detection region, and a fluid ejector fluidically coupled to the optical-detection region.

In some examples, the fluid ejector includes an ejection nozzle and a fluidic actuator disposed with an ejection chamber fluidically coupled to the optical-detection region. For example, the fluidic actuator may actuate to cause flow of the fluid from the reservoir to the cell-poration region and to the optical-detection region and to eject the cell from the microfluidic device through the ejection nozzle.

In some examples, the cell-poration mechanism includes a set of electrodes. In some examples, the cell-poration region includes a microfluidic channel that is serpentine-shaped and with the set of electrodes extending therethrough at a plurality of portions of the microfluidic channel.

In some examples, the cell-poration mechanism includes a constriction portion. In some examples, the cell-poration region includes a microfluidic channel with the constriction portion that includes a circumference that is attenuated from remaining portions of the microfluidic channel.

In some examples, the cell-poration mechanism further includes a set of electrodes including a first electrode disposed upstream of the constriction portion and a second electrode disposed downstream of the constriction portion.

In some examples, the device further includes a first sensor region disposed between the cell-poration region and the optical-detection region, the first sensor region including a first microfluidic channel with a first sensor disposed with the first microfluidic channel. In some example, the device further includes a second sensor region disposed between the reservoir and the cell-poration region, the second sensor region including a second microfluidic channel with a second sensor disposed with the second microfluidic channel.

Various examples are directed to an apparatus comprising a microfluidic device, an optical sensing device, and circuitry. The microfluidic device includes a reservoir to store a fluid containing at least a cell and at least a molecular probe, a cell-poration region fluidically coupled to the reservoir and including a set of electrodes, an optical-detection region fluidically coupled to the cell-poration region and including an optically transparent window associated with a wall of the optical-detection region, and a fluid ejector fluidically coupled to the optical-detection region. The apparatus further includes an optical sensing device to measure emitted light responsive to excitation light provided toward the optical-detection region, and circuitry communicatively coupled to the microfluidic device and the optical sensing device to drive flow of the fluid through the microfluidic device via the fluid ejector and to detect binding of the molecular probe to an intracellular target in the cell via the measured emitted light.

In some examples, the apparatus further includes a substrate including a plurality of regions and a stage coupled to the substrate, wherein the circuitry is communicatively coupled to the stage to instruct the stage to move the substrate relative to the fluid ejector, such that the fluid ejector is aligned with a select region of the plurality of regions of the substrate. And the fluid ejector is to selectively eject a fluid droplet of the fluid containing the cell from the microfluidic device to the select region of the plurality of regions of the substrate.

In some examples, the fluid includes a plurality of cells including the cell and a plurality of the molecular probe, and the circuitry is to assess for a presence of the intracellular target in the plurality of cells via the measured emitted light associated with each of the plurality of cells, and actuate the fluid ejector to cause selective ejection of the plurality of cells from the microfluidic device to a plurality of regions of the substrate and based on the assessed presence.

In some examples, the cell-poration region further includes a microfluidic channel with a constriction portion, wherein a first electrode of the set of electrodes is disposed upstream of the constriction portion and a second electrode of the set of electrodes is disposed downstream of the constriction portion.

Some examples are directed to a method comprising flowing a fluid containing at least a cell and at least a molecular probe from a reservoir to a cell-poration region and to an optical-detection region of a microfluidic device via actuation of a fluid ejector and while the cell is exposed to the molecular probe, porating the cell using a cell-poration mechanism in the cell-poration region (and while the cell is exposed to the molecular probe), providing excitation light toward an optically transparent window associated with a wall of the optical-detection region using an optical sensing device, assessing for a presence of an intracellular target in the cell using light emitted from the optical-detection region in response to the excitation light and associated with the cell, and selectively ejecting the cell from the microfluidic device to a substrate based on the assessment and using the fluid ejector.

In some examples, porating the cell includes forming apertures in the cell membrane of the cell by flowing the cell through the cell-poration region. Wherein the cell-poration mechanism includes at least one of a constriction portion and a set of electrodes disposed with a microfluidic channel, wherein porating includes at least one of: mechanical porating the cell by flowing the cell through the constriction portion, and electroporating the cell by an electric field applied across the cell via the set of electrodes of the cell-poration region. And, the method further includes incubating the cell with the apertures with the molecular probe such that the molecular probe is allowed to pass through one of the apertures and bind to the intracellular target of the cell.

In some examples, the fluid includes a plurality of cells including the cell of interest and a plurality of the molecular probe (e.g., a volume of the molecular probe), the method further including selectively ejecting the plurality of cells by: in response to detecting the presence of the intracellular target, ejecting respective cells of the plurality to select ones of a plurality of regions of the substrate, and in response to not detecting the presence of the intracellular target, ejecting respective cells of the plurality to a waste region of the plurality of regions of the substrate.

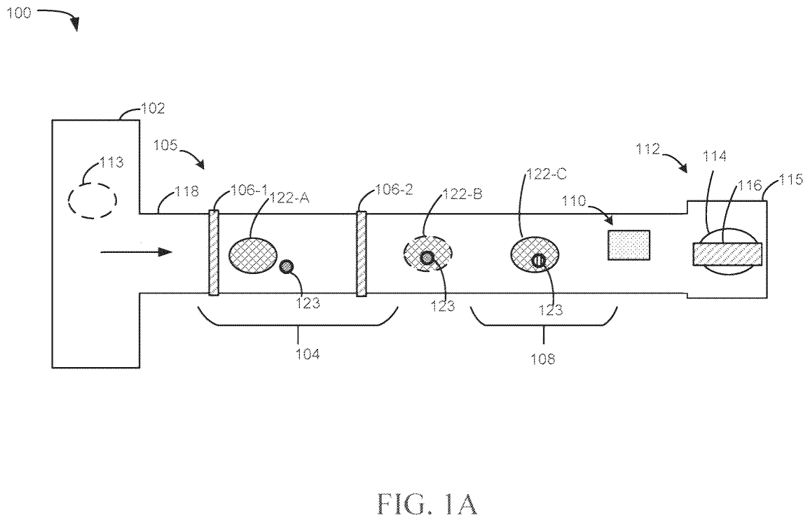

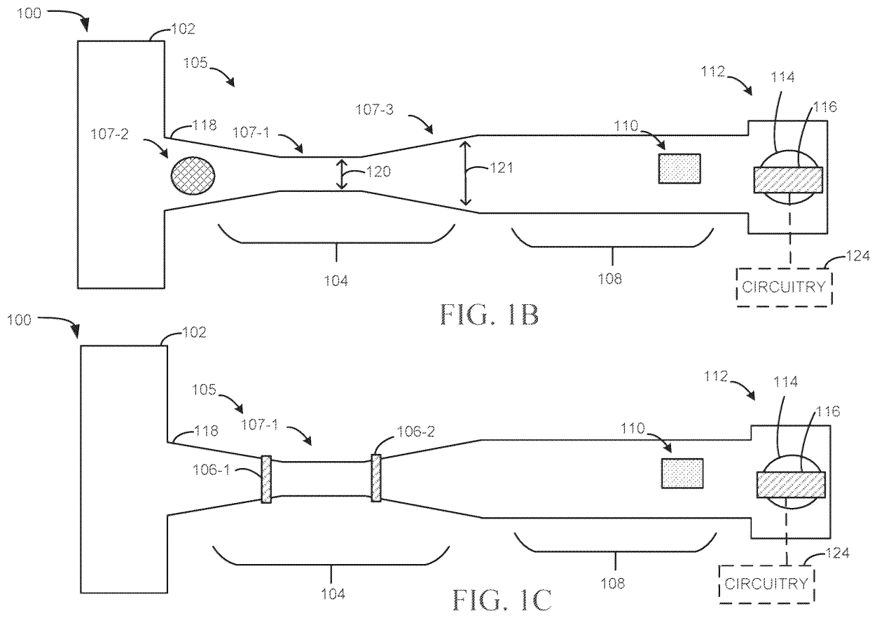

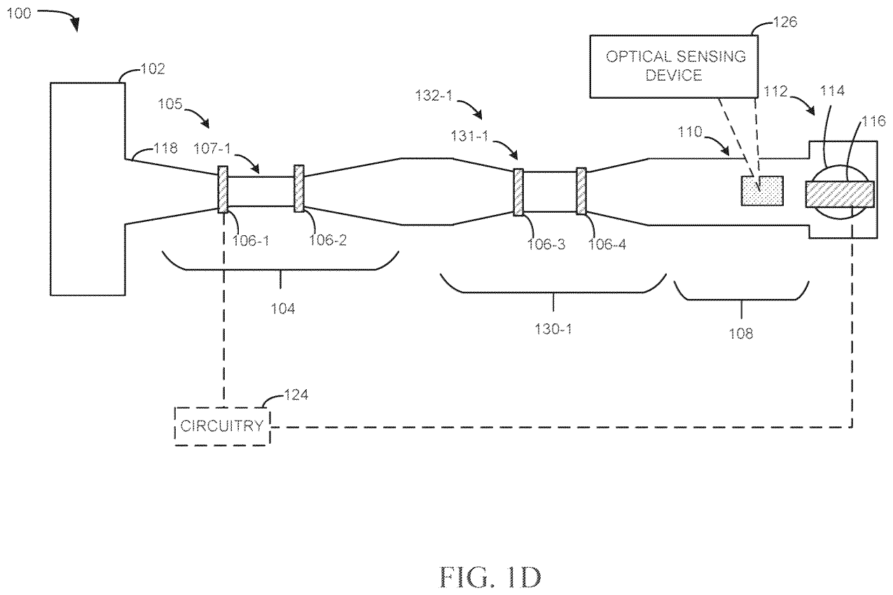

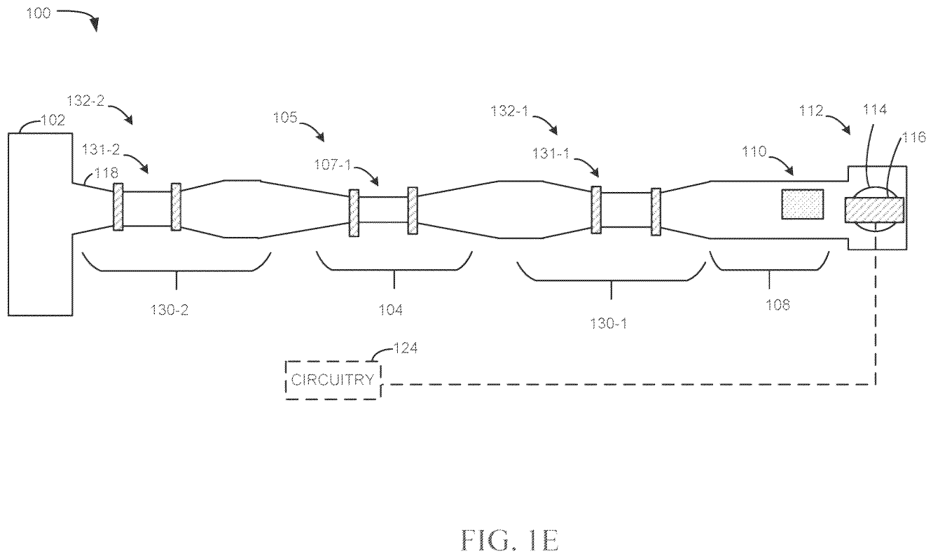

Turning now to the figures, FIGS. 1A-1E illustrate example microfluidic devices with a cell-poration region, optical-detection region, and fluid ejector, in accordance with the present disclosure. Any of the microfluidic devices illustrated by FIGS. 1A-1E may be used to flow a fluid therethrough, with the fluid containing cells and at least a molecular probe. At least some of the cells may include an intracellular target which the molecular probe is designed to bind to.

Referring to FIG. 1A, the microfluidic device 100 includes a reservoir 102, a cell-poration region 104, an optical-detection region 108, and a fluid ejector 112. A microfluidic device 100 may include reservoirs, chambers, and/or channels formed by and/or between substrates as etched or micromachined portions. For examples, the chamber(s), wells, and/or channels may be defined by surfaces fabricated in the substrate(s) of the microfluidic device 100. As shown by FIG. 1A, the cell-poration region 104, optical-detection region 108, and fluid ejector 112 may be disposed along or form part of a microfluidic channel 118 which is coupled to the reservoir 102. As used herein, a microfluidic channel refers to or includes a path through which a fluid or semi-fluid may pass, which may allow for transport of volumes of fluid on the order of μL, nanoliters, picoliters, or femtoliters. A chamber refers to or includes an enclosed and/or semi-enclosed region of the microfluidic device 100, which may be used to perform chemical processing on fluids therein.

The reservoir 102 may contain a fluid including at least a cell and at least a molecular probe. As used herein, a reservoir refers to or includes a column capable of storing a volume of fluid. The reservoir 102 may be sized and shaped to hold the fluid while a portion of the fluid flows through other portions of the microfluidic device 100. In some examples, the reservoir 102 may contain a volume of fluid between about 1 microliter (μL) to about 10 milliliters (mL). In some examples, the reservoir 102 may contain a volume of fluid between about 10 μL to about 10 ML, about 10 μL to about 500 μL, about 200 μL to about 800 μL, about 500 μL to about 1 mL, about 1 mL to about 10 mL, about 2 mL to about 4 mL, among other ranges. In some examples, the reservoir 102 may have a height and length ranging from about 1 millimeter (mm) by about 1 mm to about 10 mm by about 10 mm, or from about 2 mm by about 2 mm to about 8 mm by about 8 mm. In some examples, the reservoir 102 may have a width ranging from about 0.3 mm to about 5 mm, about 0.5 mm to about 2.5 mm, about 0.5 mm to about 2.5 mm, about 1 mm to about 5 mm, about 1 mm to about 3 mm, or about 2 mm to about 4 mm. The reservoir 102 may have a cross-sectional area larger than a cross-sectional area of other portions of the microfluidic device 100, such as the cell-poration region 104 and/or optical-detection region 108.

A molecular probe, as used herein, refers to or includes molecules or compounds that bind to an intracellular target, and in response, provides a detectable optical signal. For example, the molecular probe may include a complex of nucleic acids, proteins, protein fragments, and/or nanoparticles coupled with a detectable moiety, molecule, or compound that exhibits the optical signal, such as a fluorescent moiety. In some examples, the molecular probe includes a nucleic acid or complex of nucleic acids that encodes for a protein and is coupled to the detectable moiety, molecule, or compound exhibiting the optical signal. An optical signal refers to or includes an electromagnetic signal or energy wave. The molecular probes may react or bind to the intracellular target, and in response, exhibit the optical signal, such as a particular fluorescent signal. As the optical signal is exhibited in response to binding with the intracellular target, the intracellular target may be detected without the use of a wash step to remove unbound molecular probes. Example molecular probes include and/or may be selected from a group consisting of a linear fluorescence resonance energy transfer (FRET) probe, a molecular beacon, a linear oligonucleotide (ODN) probe, dual FRET donor and acceptor beacons, an autoligation FRET probe, a probe coated with a bacterial phage MS2 fused with a fluorescent protein, and/or a probe with a fragment complementation of the fluorescent protein, and combinations thereof. In some examples, linear FRET probes or molecular beacons may be used due to ease of manufacturing and optically sensing the probes as compared to other probes. However, examples are not so limited.

Linear FRET probes include two linear oligonucleotides that are fluorescently labeled at a 5′ and a 3′ end with donor and acceptor fluorophores, respectively, to form a FRET pair. A donor fluorophore is a fluorophore that is initially excited by light and may transfer energy to the acceptor fluorophore. The acceptor fluorophore is a fluorophore that receives the transferred energy. The two oligonucleotides are designed to hybridize to adjacent regions on the intracellular target, e.g., target nucleic acid, such that the donor and acceptor fluorophores are brought into close proximity when both oligonucleotides are hybridized to the same intracellular target. Excitation of donor fluorophore is followed by nonradiative energy transfer from it to the acceptor fluorophore on the other oligonucleotides, when both are in close proximity to each other, such as being closer than about 10 nanometers (nm).

Molecular beacons are hair pin oligonucleotides labeled at one end with a reporter fluorophore and at the other end with a quencher. A reporter fluorophore is a fluorophore, e.g., a molecule or compound, that emits a particular fluorescent signal. A quencher is a molecule or compound that suppresses or reduces the fluorescent signal of the reporter fluorophore when the quencher and reporter are proximate to one another. The molecular beacons have a complimentary sequence region near the ends and a hairpin region in the center. The complimentary regions hybridize in the absence of a target, bringing the fluorophore and quencher together which renders the beacon non-fluorescent. In the presence of the intracellular target, the beacon opens up, with the center hairpin region binding to the target, and not allowing the end regions to be next to each other, thus preventing quenching from occurring. This renders the beacon fluorescent.

A linear ODN probe includes a fluorescently labeled oligonucleotide that is complementary to the intracellular target, and may include a fluorophore that provides a detectable fluorescent signal. In various examples, a volume of the linear ODN probe is used such that the fluorescent signal is above a background signal of unbound probes. To detect the intracellular target, multiple linear ODN probes may bind to at least one intracellular target within the cell membrane. The linear FRET probes, described above, may include two linear ODN probes.

Dual FRET donor and acceptor beacons combine the concepts of the linear FRET probes with the molecular beacons. More particularly, there is a donor molecular beacon labeled with a donor fluorophore at one end (5′ or 3′ end) and with a quencher at the other end (3′ or 5′ end) and an acceptor molecular beacon labeled with an acceptor fluorophore at one end (3′ or 5′ end) and with a quencher at the other end (5′ or 3′ end). Each of the donor and acceptor molecular beacons have a complimentary sequence region near the ends and a hairpin region in the center. The complimentary regions hybridize in the absence of an intracellular target, bringing the (donor or acceptor) fluorophore and quencher together which renders the beacon non-fluorescent. Each of the donor and acceptor molecular beacons are designed to bind to a different portion of the intracellular target. In the presence of the intracellular target, each beacon opens up, with the center hairpin region binding to the intracellular target, and not allowing the end regions to be next to each other, thus preventing quenching from occurring. The center hairpin regions of the molecular beacons are designed to hybridize to adjacent regions on the intracellular target, such that the donor and acceptor fluorophores are brought into close proximity when both beacons are hybridized to the same intracellular target. Excitation of donor fluorophore is followed by nonradiative energy transfer from the donor fluorophore to the acceptor fluorophore on the other oligonucleotide, when both are in close proximity to each other, such as being closer than about 10 nm.

Autoligation FRET probes include two linear oligonucleotides that are fluorescently labeled at a 5′ and a 3′ end with donor and acceptor fluorophores. The oligonucleotide with the donor fluorophore includes a quencher disposed proximate to the donor fluorophore, such the donor fluorophore is quenched when the two oligonucleotides are not bound to the intracellular target. The two linear oligonucleotides hybridize to adjacent regions on the intracellular target, and in response, the quencher is displaced and ligation brings the donor and acceptor fluorophores into close proximity.

A probe coated with a bacterial phage MS2 fused with a fluorescent protein, such as green fluorescent protein (GFP), red fluorescent protein (RFP), and yellow fluorescent protein (YFP), among others, may be used in some examples. The MS2-fluorescent protein complex binds to multiple hairpin sequences in 3′ untranslated region of messenger RNA (mRNA), which provides a fluorescent signal that is higher than the background signal.

Probes with a fragment complementation of a fluorescent protein include two RNA-binding proteins, which each carry a fragment of the fluorescent protein. The two RNA-binding proteins bind to adjacent regions on the RNA sequence such that the two fragments of the fluorescent protein are brought together to activate the fluorescent signal.

The optical signal, e.g., fluorophore, may be provided by a fluorescent moiety and/or protein. Example fluorescent moieties and/or proteins include fluorescent proteins, quantum dots, and organic dyes. Examples are not limited to fluorophores and may include other optical signals, such as luciferase. Non-limiting examples of optical signals include GFP, RFP, YFP, phycoerythrin, allophycocyanin, luciferase, fluorescein, Dylight 649, Alexa647, and Alex750, among others and in various combinations thereof.

Although the above describes the example molecular probes as binding to intracellular targets of nucleic acids, examples are not so limited. For example, a molecule probe may bind to other molecules, such as proteins, lipids, and non-biologic compounds that are less than 800 daltons, sometimes referred to as “a small molecule”. For example, the molecular probe may be an aptamer. An aptamer is an oligonucleotide that folds into a three-dimensional shape to bind non-covalently to the target small molecule. Example small molecules include toxins, antibiotics, drugs, and heavy metals. In some examples, in response to binding to the small molecule, the aptamer may change shape and thus change a FRET response or otherwise provide an optical signal. Further, examples are not limited to molecular probes which include nucleic acids, and may include molecular probes that include a protein, protein fragment, or nanoparticle, such as the above-described MS2-fluorscent protein complex and RNA-binding proteins, enzymes, or ribonucleoprotein complexes, among others.

The microfluidic device 100 may pass a portion of a fluid, which include at least a cell and at least a molecular probe. The fluid may be passed via the fluid ejector 112 that actively moves fluid from the reservoir 102 into the cell-poration region 104, and into the optical-detection region 108, as shown by the arrow and as described below.

In some examples, the fluid may include a biological sample containing cells that is mixed with a medium and/or a buffer including the molecular probes, and loaded into the reservoir 102. For example, the fluid may contain of plurality of molecular probes of the same type. In some examples, the fluid may contain multiple types of probes. In other examples, the biological sample and molecular probes may be inserted separately and mix in the reservoir 102. In some examples, the reservoir 102 may include or be associated with a fluid inlet 113. The fluid inlet 113 may include an aperture in the microfluidic device 100 and/or puncturable material that permits loading of the fluid containing the cell and/or the molecular probe to the reservoir 102. A fluidic inlet refers to or includes an inlet port, e.g., an aperture, of the microfluidic device 100.

Fluid flow may be caused by actuation of the fluid ejector 112 that is internal to the microfluidic device 100. As used herein, a fluid ejector refers to or includes a physical structure, such as an ejection chamber 115, to receive a fluid, such as from a manifold, fluid slot, or fluid hole array. For example, the fluid ejector 112 may include an ejection nozzle 114 and a fluidic actuator 116 disposed in an ejection chamber 115. An ejection chamber refers to or includes a semi-enclosed region of the microfluidic device 100, with the ejection nozzle 114 and fluidic actuator 116 disposed within and/or through a surface of the ejection chamber 115. The fluidic actuator 116 may be disposed in the ejection chamber 115 coupled to the ejection nozzle 114 and the optical-detection region 108, and is positioned in line with the ejection nozzle 114. For instance, the fluidic actuator 116 may be positioned directly above or below the ejection nozzle 114. A fluidic actuator, as used herein, refers to or includes circuitry and/or a physical structure that causes movement of fluid. The fluidic actuator 116 may be fired or pulsed, which creates the fluid flow by pushing or pulling fluid within the regions 104, 108 of the microfluidic device 100. Actuation of the fluidic actuator 116 may cause some fluid contained in the optical-detection region 108 to be dispensed or expelled out of the ejection nozzle 114. The fluidic actuator 116 may be actuated via application of a voltage or current by circuitry, as further described below.

Example fluidic actuators include electrodes, a fluidic pump, a magnetostrictive element, an ultrasound source, mechanical/impact driven membrane actuators, and magneto-restrictive drive actuators, among others. Example fluidic pumps include a piezo-electric pump and a resistor, such as a thermal inkjet resistor (TIJ).

In some examples, the fluidic actuator 116 includes a TIJ resistor. Activation of the TIJ resistor may create the flow of fluid by firing drops of fluid from the microfluidic device 100 and/or creating a vapor bubble. As a specific example, the resistor may be actuated to cause firing of drops of fluid from the ejection nozzle 114, which creates the fluid flow through the microfluidic device 100 by pulling the fluid toward the ejection nozzle 114. The TIJ resistor may create bubbles that force the fluid droplets of fluid out of the ejection chamber 115. For example, a pulse of current may be passed through the fluidic actuator 116 of fluid ejector 112 in the form of a TIJ resistor positioned in fluid ejector 112. The TIJ resister acts as a heater, and heat from the TIJ resistor causes vaporization of fluid in the ejection chamber 115 of the fluid ejector 112 to form the vapor bubble, which causes a pressure increase that propels the fluid droplet of fluid.

However, examples are not so limited and a variety of different types of resistors may be used. In some examples, the fluidic actuator 116 includes a piezoelectric-based pump. The piezoelectric-based pump may generate pressure pulses that force fluid droplets of the reaction fluid out of the ejection nozzle 114. In such piezoelectric-based pumps, a voltage may be applied to the fluidic actuator 116 that is in the form of a piezoelectric element (e.g., piezoelectric material) located in the ejection chamber 115 of the fluid ejector 112. When a voltage is applied, the piezoelectric element changes shape, which generates a pressure pulse that forces a fluid droplet of the reaction fluid from the fluid ejector 112.

In some examples, the ejection nozzle 114 is an orifice located near the fluidic actuator 116. In some examples, the fluidic actuator 116 may be first actuated to direct the flow of the cell through the microfluidic device 100 and actuated a second time to eject the cell from the microfluidic device 100. For example, the fluidic actuator 116 may act as the fluidic pump to pull fluid including the cell, as shown by 122-A, 122-B, 122-C, to and through cell-poration region 104 and the optical-detection region 108 and eject fluid from the ejection nozzle 114. The ejection nozzle 114 may be defined by a surface of the ejection chamber 115, through which fluid is ejected in drops for further processing.

As an example, a cell may be carried along the microfluidic device 100 by the fluid flow in response to firing or pulsing the fluidic actuator 116, with the number of fired drops to move the cell through the microfluidic device 100 to the fluid ejector 112 varying depending on the flow path, as shown by the movement of the cell illustrated by 122-A, 122-B, 122-C. The fluid flow may cause the cell to travel from the reservoir 102 into the cell-poration region 104, as shown by 122-A, through the cell-poration region 104, as shown by 122-B, and into the optical-detection region 108, as shown by 122-C. In response to traveling through the cell-poration region 104, as further described below, apertures are formed and the cell with the apertures may be incubated with the molecular probe 123 and allowed to recover from the electric field and/or shear force applied via the cell-poration region 104. The molecular probe 123 may be smaller in circumference than the apertures, and may pass through an aperture of the cell membrane and into the intracellular space of the cell. As the cell recovers over time, the apertures close and trap the molecular probe 123 within the cell, as shown by 122-C. The flow rate may be dependent on a firing or pulse rate of fluidic actuator 116, which may be adjusted to set the flow rate and set the electric field and/or shear force applied to the cell.

Although examples describe porating a cell, examples are not so limited. In some examples, a plurality of cells are porated and transfected or transformed at the same time and/or using multiple processes along microfluidic channel 118. For example, the reservoir 102 may store a biologic sample or other fluid containing a plurality of cells.

The cell-poration region 104 is fluidically coupled to the reservoir 102 and includes a cell-poration mechanism 105. The cell-poration region includes a region of the microfluidic device 100 in which cell poration is performed. The cell-poration region 104 may include a microfluidic channel or a chamber, such as a portion of microfluidic channel 118. A cell-poration mechanism refers to or includes hardware used to form apertures in cell membrane of the cell.

In some examples, as illustrated by FIG. 1A, the cell-poration mechanism 105 includes a set of electrodes 106-1, 106-2. The set of electrodes 106-1, 106-2 may be used to perform electroporation on the cell. For example, the cell may be flown through the cell-poration region 104 and the set of electrodes 106-1, 106-2 may be actuated or otherwise have a potential applied that results in an electric field being applied across the cell as shown by 122-A, 122-B, and 122-C. To electroporate the cell, a threshold electric field may be applied for a threshold period of time to cause apertures to form in the cell membrane that are larger than the molecular probe 123. In some examples, the potential and electric field applied may be between about 10 kilohertz (kHz) and about 20 millihertz (mHz), between about 0.07 and about 0.2 volts per meter (V/um) root mean square (RMS) and for between about 0.1 milliseconds (ms) and about 1000 ms. In some examples, the potential and electric field applied may be about 200 KHz and between about 0.07 V/um RMS and about 0.2 V/um RMS and for between about 10 ms and about 100 ms. The energy and/or current applied may be dependent on electrode size and conductivity of the fluid. The conductivity of the fluid may be between about 0.001 siemens per meter (S/m) and about 2 S/m, or about 1.5 S/m. The electric field may cause the apertures to form in the cell membrane, as shown by 122-B, and the molecular probe 123 may enter or pass through an aperture and bind to an intracellular target. As cell moves out of the cell-poration region 104, the electric field is removed from being applied across the cell and the apertures in the cell membrane may close, thereby trapping the molecular probe 123 in the cell, as shown by 122-C. The cell may be allowed to incubate with the molecular probe 123 to allow for binding. Electroporation may allow for porating cells of greater sizes and may allow for greater control of poration and/or transfection or transformation with reduced clogging and decreased cell viability as compared to other poration techniques, such as mechanical poration. For example, electroporation may allow for transfecting or transforming cells of about 12-15 μm in diameter, which may be mammalian cells, and cells that are larger, such as cells that are greater than 20 μm in diameter.

As described above, for electroporation, the flow of the cell through the electric field applied by the set of electrodes 106-1, 106-2 may cause the cell to form apertures in the cell membrane sufficient for the molecular probe to transition through. Cells of different sizes and/or apertures of different diameters may be formed by adjusting an exposure time to the electric field field, a magnitude of the electric field, a frequency of the electric field, buffer conductivity, and/or buffer composition (e.g., inclusion of dimethylsulfoxide (DMSO), for example, may alter membrane fluidity and help with electroporation). In some examples, the exposure time may be adjusted by adjusting a flow rate, adjusting the length of the cell-poration region 104, and/or adjusting the time the electrodes 106-1, 106-2 are actuated. The magnitude and/or frequency of the electric field may be adjusted by the type or size of electrodes used and/or by selective actuation of the electrodes 106-1, 106-2.

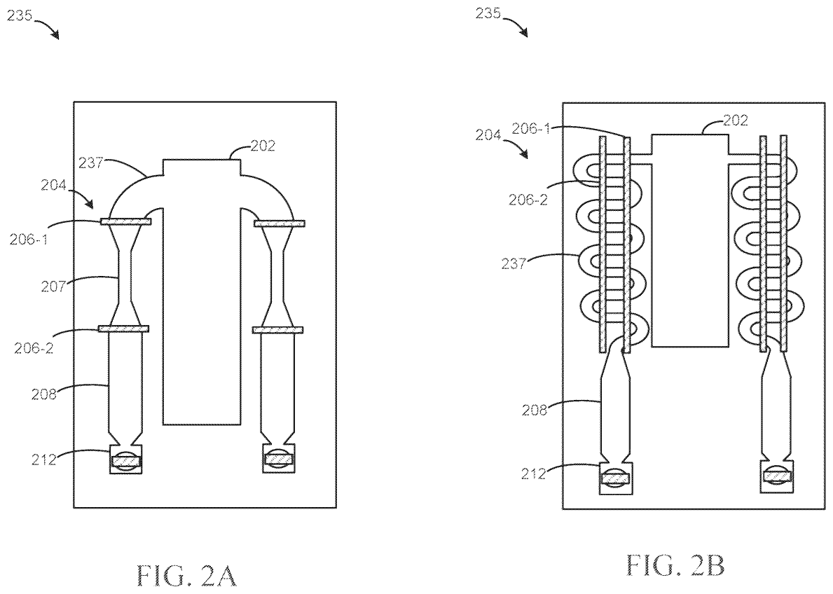

In some examples, as illustrated by and referring to FIG. 2B, the cell-poration region 204 includes a microfluidic channel 237 (or a portion of microfluidic channel 118 of FIG. 1A) that is serpentine-shaped and with the set of electrodes 206-1, 206-2 extending therethrough at a plurality of portions of the microfluidic channel 237. The serpentine-shaped channel 237 may allow for multiple applications of the electric field to be applied to the cell, with periods of rest for the cell between the applications of the electric field. By cycling between applications of the electric field and no electric field applied across the cell, the amount of time the cell is exposed to the electric field may be increased while minimizing issues with cell viability as compared to one application of the electric field to generate the apertures in the cell membrane of sufficient size to introduce a molecular probe within the cell. For example, application of one electric field may include a larger magnitude electric field applied for a shorter amount of time to generate apertures as compared to use of the serpentine-shaped channel 237, and the larger electric field may lyse the cell. The serpentine-shaped channel 237 may further allow for higher throughput and reduced chance of clogging as compared to other shaped channels and/or poration techniques.

In some examples, as illustrated by and referring to FIG. 1B, the cell-poration mechanism 105 includes a constriction portion 107-1 used to perform mechanical poration on the cell. For example, the cell-poration region 104 includes the microfluidic channel 118 or a portion thereof with the constriction portion 107-1 that includes a circumference that is attenuated from remaining portions 107-2, 107-3 of the microfluidic channel 118. As shown, the constriction portion 107-1 includes a first circumference 120 that is attenuated from remaining portions 107-2, 107-3 of the microfluidic channel 118, as illustrated by the second circumference 121 of the microfluidic channel 118 which is larger than the first circumference 120. Although the arrows showing the first circumference 120 and the second circumference 121 are illustrated as linear, the first circumference 120 and the second circumference 121 are along the perimeter of the constriction portion 107-1 and the remaining portions 107-2, 107-3 of the cell-poration region 104.

The constriction portion 107-1 has a circumference that is attenuated from remaining portions 107-2, 107-3 of the cell-poration region 104, thereby increasing flow velocity through the constriction portion 107-1 from the remaining portions 107-2, 107-3. As used herein, a circumference of the constriction portion or other portions of the cell-poration region 104 refers to or includes a distance around a perimeter of the constriction portion or portion of the cell-poration region 104. A circumference that is attenuated, as used herein, refers to or includes a reduced circumference as compared to the circumference of the remaining portions of the microfluidic channel 118. In some examples, the constriction portion 107-1 with the attenuated circumference may have a hydraulic diameter that is smaller than the hydraulic diameter of the remaining portions 107-2, 107-3 of the cell-poration region 104 or the microfluidic channel 118. In some examples, the hydraulic diameter of the constriction portion 107-1 may be smaller than the nominal diameter of the cell. In some examples, a constriction portion 107-1 may be a portion of the microfluidic channel 118 that has an oblong cross-sectional shape, such that a diameter along the width is different than a diameter along the height of the cell-poration region 104. In some examples, a transection of the circumference of the constriction portion 107-1 may be smaller than the nominal diameter of the cell, such that the cell is deformed when travelling through the constriction portion 107-1. A transection of the circumference refers to or includes a cross-section or a slice of the constriction portion 107-1 taken at a point along the length of the constriction portion 107-1. The flow of the cell through the constriction portion 107-1 may cause the cell to form apertures in the cell membrane sufficient for the molecular probe to transition through.

For mechanical poration, and similarly to that described above, the microfluidic channel 118 may pass a portion of the fluid, which may include a cell through the constriction portion 107-1 of the cell-poration region 104, and thereby cause formation of apertures in the cell membrane. A flow rate of the cell may be used to apply a set shear force to cause the apertures in the cell membrane to form. As used herein, a shear force refers to or includes a force caused by parallel forces acting on the cell in opposite directions and at different parts of the cell. For example, the cell may contact a wall of the constriction portion 107-1 while flowing through the cell-poration region 104, which may cause a pulling force on the cell in the direction of the fluid flow and a pulling force on the cell in the opposite direction of the fluid flow. The shear force applied may be set to cause apertures of a sufficient size for a particular molecular probe to pass through. Too much shear force may result in cell lysis or death, and too little shear force may result in no apertures or apertures of an insufficient size for the molecular probe to pass through (e.g., for transfection or transformation, or otherwise introducing the molecular probe within the cell). In some examples, the first circumference 120 of the constriction portion 107-1 may constrict the cell from ninety % to thirty % of a nominal diameter of the cell, however examples are not so limited.

In some examples, compression, tensile, and shear forces are applied on the cell by flowing into and through the constriction portion 107-1. As used herein, a compression force refers to or includes a force caused by opposing forces that push one part of the cell in a first direction and push another part of the cell in a second and opposite direction. For example, a compression force may squeeze or decrease a length of the cell, and may be caused by the cell entering the constriction portion 107-1. A tensile force refers to or includes a force caused by opposing forces that pull one part of the cell in a first direction and pull another part of the cell in a second and opposite direction. For example, the tensile force may stretch or lengthen the cell, and may be caused by the cell exiting the constriction portion 107-1. As described above, a shear force may be applied to the cell due to parallel forces acting on the cell from the cell contacting the wall of the constriction portion 107-1 while being flown through the constriction portion 107-1, which causes shear tension due to the parallel forces in the direction of the flow and opposite to the direction of the flow.

The amount of forces applied may be adjusted based on the initial flow rate of the fluid, a degree of attenuation, and dimensions of the constriction portion 107-1 and the remaining portions 107-2,107-3 of the cell-poration region 104 including circumferences and/or lengths. For example, the remaining portions 107-2,107-3 of the cell-poration region 104 may be respectively upstream and downstream from the constriction portion 107-1 and may include the second circumference 121 that is larger than the first circumference 120 of the constriction portion 107-1. The size and length of the remaining portions 107-2,107-3 of the cell-poration region 104 may impact a degree and duration of forces from acceleration and/or deceleration as the cell enters into and/or exits from the constriction portion 107-1 of the cell-poration region 104. The first circumference 120 may impact a maximum flow rate and friction forces between the walls of the constriction portion 107-1 and the cell membrane, and the length may impact the amount of time that the compression force, tensile force, and shear force is applied to the cell. The degree of attenuation may refer to or include a change in circumference between the constriction portion 107-1 and the remaining portions 107-2,107-3 and/or within the constriction portion 107-1, such as a taper of circumference transitions between the constriction portion 107-1 and the remaining portions 107-2, 107-3 and which may cause changes in forces applied on the cell and/or changes in the flow rate when the cell is flowing within the constriction portion 107-1 and between or within the remaining portions 107-2, 107-3.

In some examples, as illustrated by and referring to FIG. 1C, the cell-poration mechanism 105 further includes a set of electrodes 106-1, 106-2. For example, the cell-poration mechanism 105 includes a first electrode 106-1 disposed upstream of the constriction portion 107-1 and a second electrode 106-2 disposed downstream of the constriction portion 107-1. In such examples, the cell-poration region 104 may perform both electroporation and mechanical poration for introducing the molecular probe within the cell, e.g., transfecting or transforming the cell. For examples, an electric field may be applied across the cell-poration region 104 as a cell flows therethrough using the first and second electrodes 106-1, 106-2, as previously described in connection with FIG. 1A, and shear forces (among other forces) may be applied to the cell as the cell flows into and through the constriction portion 107-1, as previously described in connection with FIG. 1C.

Referring back to FIG. 1A, the microfluidic device 100 further includes an optical-detection region 108 that is fluidically coupled to the cell-poration region 104. The optical-detection region 108 includes a region of the microfluidic device 100 in which optical interrogation of fluid within the microfluidic device 100 is performed. The optical-detection region 108 may include a microfluidic channel and/or a chamber, such as a portion of microfluidic channel 118. The optical-detection region 108 includes an optically transparent window 110 associated with a wall of the optical-detection region 108. The optically transparent window 110 may include a portion of the wall or the whole wall that is optically transparent, such that light may travel through and is used to detect binding between the molecular probe and the intracellular target. As used herein, optically transparent refers to or includes a material of the portion of the wall or the wall that permits at least about ninety percent (%) of a wavelength of light within an emission range of a light source or a detection range of an optical detector to pass through and into the microfluidic device 100. In some examples, the optical-detection region 108 may be constructed from an optically transparent material and may be side illuminated. For example, the optically transparent window 110 may permit for the detection of a cell passing there through by allowing excitation light to enter the optical-detection region 108 and excite the molecular probe bound to the intracellular target within the cell. Based on the fluorescent signal emitted, binding of the molecular probe to the intracellular target is detected and/or a cell type of the cell may be determined.

As noted above, the microfluidic device 100 further includes a fluid ejector 112 fluidically coupled to the optical-detection region 108. As previously described, the fluid ejector 112 includes an ejection nozzle 114 and a fluidic actuator 116 disposed with an ejection chamber 115 fluidically coupled to the optical-detection region 108. The fluidic actuator 116 is to actuate to cause flow of the fluid from the reservoir 102 to the cell-poration region 104 and to the optical-detection region 108 and to eject the cell from the microfluidic device 100 through the ejection nozzle 114.

In some examples, the optically transparent window 110 may be on the same wall of the microfluidic device 100 as the fluid ejector 112, and in other examples may be on a different wall. For example, the optically transparent window 110 may be located on or form part of a wall of the microfluidic device 100 that is a 90 degree angle (or other angle) from the ejection nozzle 114 of the fluid ejector 112.

In various examples, as shown by and referring to FIGS. 1B-1C, the microfluidic device 100 may include or is couplable to circuitry 124. The circuitry 124 may selectively actuate the fluidic actuator 116 to drive flow of the fluid through the microfluidic device 100 and to detect binding of the molecular probe to an intracellular target in the cell via the measured emitted light. The circuitry 124 may actuate the fluidic actuator 116 to set the flow rate of the fluid through the cell-poration region 104, as further described herein. In some examples, the circuitry may actuate electrodes 106-1, 106-2 as shown by FIG. 1C to provide the electric field and perform electroporation.

In some examples, the fluid ejector 112 may be actuated a plurality of times to direct the flow. For example, the fluid ejector 112 may be actuated a first time to direct flow of a portion of the fluid from the reservoir 102 to the cell-poration region 104, and actuated a second time to direct the flow of the portion of the fluid through the cell-poration region 104 and to the optical-detection region 108. The fluid ejector 112 may be actuated a third time to eject the portion of the fluid out of the microfluidic device 100 through the ejection nozzle 114. In some examples, directing the flow of the cell through the cell-poration region 104, such as through the constriction portion 107-1, includes changing a flow of a portion of the fluid from a first velocity to a second velocity, thereby flowing the cell into the cell-poration region 104 and causing electrical and/or mechanical poration for introducing the molecular probe within the cell (e.g., transfecting or transforming the cell).

For example, the fluid ejector 112 may be fired or pulsed a plurality of times. Each pulse of the fluid ejector 112 may move a volume of fluid including five to one hundred fifty picoliters (pL), and the fluid ejector 112 may be fired at speeds of up to 10,000 to 50,000 hertz (Hz), such as between about 0.1 kHz to about 50 kHz or about 0.5 kHz. The volume of fluid within the cell-poration region 104 may be one to ten pL, such that one pulse of the fluid ejector 112 may transport the cell through the cell-poration region 104. However, examples are not so limited.

In some examples, the fluid ejector 112 is actuated one time, and which directs the flow of the portion of the fluid from the reservoir 102 to and through the cell-poration region 104 and to the optical-detection region 108. The flow rate may be set to provide a set electrical force and/or shear force for a particular type of cell when the cell travels through the cell-poration region 104.

Microfluidic devices of the present disclosure integrate porating cells, and transfection or transformation of cells with molecular probes, detection of an intracellular target using the molecular probes, and ejection or sorting of the cells based on the detection. The transfection or transformation of the cell is upstream of detection to allow for incubation with the molecular probe and for a resulting fluorescent signal to be activated in response to binding between the molecular probe and the intracellular target. The fluid flow and ejection of fluid may be controlled by an integrated fluid ejector which digitally ejects a consistent volume of fluid with each pump. The fluid flow may be temporarily stopped or slowed while a cell is within the optical-detection region to allow for incubation and/or detection. By having controlled fluid flow, ejection, and time for detection, cells may be sorted based on intracellular targets and while maintaining cell viability and without a wash step, thus allowing for further processing on the cells and/or culturing of the cells.

The microfluidic device 100 illustrated by FIG. 1A may include variations, some of which are illustrated by FIGS. 1B-1E. The variations may include, but are not limited to, cell-poration regions including a constriction portion or both electrodes and a constriction portion, coupled circuitry, and the addition of sensor regions, among others. Each of the microfluidic devices 100 of FIGS. 1B-1E include an implementation of the microfluidic device 100 of FIG. 1A, including at least some of substantially the same features and components, as illustrated by the common numbering. The common features and components are not repeated for ease of reference.

FIGS. 1B-1C illustrate example implementations of microfluidic device 100 that include a constriction portion 107-1 within the cell-poration region 104. As previously described, the cell-poration mechanism 105 may alternatively or additionally include a constriction portion 107-1, as compared to the cell-poration mechanism 105 illustrated by the microfluidic device 100 of FIG. 1A. FIG. 1B illustrates a cell-poration mechanism 105 including the constriction portion 107-1, and FIG. 1C illustrates a cell-poration mechanism 105 include the set of electrodes 106-1, 106-2 and the constriction portion 107-1.

In the examples of FIG. 1B-1C, circuitry 124 may actuate the fluid ejector 112 to direct the flow of the cell into and through the constriction portion 107-1 at a set flow rate. In some examples, the set flow rate may be specific to the type of cell and an amount of shear force (and/or electric field) to apply to cause apertures in the type of cell and for a type of molecular probe. The flow rate may impact the amount of shear force applied on the cell, for example, by changing an amount of time that the cell is exposed to the shear force when traveling through the constriction portion 107-1. In some examples, as described further herein, the circuitry 124 may store a list that identifies the set flow rate for the particular type of cell and/or may generate the list. Similarly and referring to FIG. 1C, the circuitry 124 may apply a particular electric field to the cell depending on the cell type, such as a strength and exposure time of the electric field. For example, by changing the strength and/or exposure time of the electric field, the size of the apertures formed may be controlled. In some examples, the exposure time may be adjusted by the set flow rate or by selectively actuating the electrodes 106-1, 106-2. The list stored by the circuitry 124 may identify the magnitude of the electric field, exposure time of the electric field, and/or flow rate for different types of cells.

In various examples, microfluidic devices may further include sensor region(s) including sensors and/or other types of sensors which may be used to sense for the presence of a cell prior to the cell-poration region or prior to the optical-detection region. FIGS. 1D-1E illustrates examples with sensor regions.

FIG. 1D illustrates an example microfluidic device 100 which includes a first sensor region 130-1 disposed between the cell-poration region 104 and the optical-detection region 108. The first sensor region 130-1 includes a first microfluidic channel 132-1 with a first sensor 131-1 disposed with the first microfluidic channel 132-1. As shown, in some examples, the first microfluidic channel 132-1 is coupled to or forms part of microfluidic channel 118. The first sensor 131-1 may include a first impedance sensor, an image sensor, a light sensor, a chemical sensor, among other types of sensor. The first sensor 131-1 may be positioned between the cell-poration region 104 and the optical-detection region 108 to detect a cell and, in response, trigger optical detection within the optical-detection region 108, sense cell viability after the electrical and/or mechanical poration is performed, and/or detect for clumps of cells or other molecules. The first sensor 131-1 may detect for a property of the cell flowing therethrough. For an impedance sensor, the buffer solution may be non-conductive, such as a phosphate buffer and/or phosphate buffered saline. As cells are conductive, an impedance detected by the impedance sensor may indicate the presence of a cell. In some examples, previously detected cells may subsequently not provide a sensor signal, which may indicate that the cell is lysed and used to optimize the set flow rates, as further described herein. For the light sensor, light scattering with the microfluidic channels may be monitored. The light sensor may be used with bacteria and small cells, which may be difficult to sense using an image sensor.

In some examples, as illustrated by FIG. 1D, the first sensor 131-1 may include an impedance sensor. The impedance sensor may measure for impedance changes in the fluid passing by. In some examples, the impedance sensor may be formed of a set of sensing electrodes 106-3, 106-4 that create an electric field within the fluid flow. As the fluid flows between the sensing electrodes 106-3, 106-4, changes in the electric field may indicate that a cell is passing through. The change in impedance may include or be associated with different cells types or with clumps of cells. For example, in response to detecting a particular cell type, the coupled optics sensing device 126 may be actuated to interrogate the cell through the optically transparent window 110 of the optical-detection region 108.

In some examples, as shown by FIG. 1E, the microfluidic device 100 further includes a second sensor region 130-2 disposed between the reservoir 102 and the cell-poration region 104. The second sensor region 130-2 includes a second microfluidic channel 132-2 with a second sensor 131-2 disposed with the second microfluidic channel 132-2. As shown, in some examples, the first and/or second microfluidic channels 132-1, 132-2 are coupled to or form part of microfluidic channel 118. The second sensor 131-2 may include a second impedance sensor, an image sensor, a light sensor, a chemical sensor, among other types of sensor. The second sensor 131-2 may be used to detect for a property of the cell flowing therethrough, and in response to trigger electroporation, to sense cell size and to optionally adjust poration parameters, such as mechanical and/or electroporation parameters. Poration parameters include parameters used to form apertures (e.g., pores) in the cell membrane of the cell, such as flow rate, electric field applied, frequency of the electric field applied, time of the electric field applied, and/or constriction portions used, as further described herein. Poration parameters may include mechanical poration parameters which may be adjusted to revise a shear (and other) force applied on the cell and/or electroporation parameters which may be revised to adjust the electric field or exposure time to the electric field. The impedance measure may indicate a size of the cell, which may be used to adjust the poration parameters and/or flow rate. For example, the type of the cell may be detected (e.g., size or other properties), and based on the type of cell, the flow rate may be adjusted to adjust the amount of electric fields and/or shear forces provided on the cell.

FIGS. 2A-2B illustrate example cell-poration regions of a microfluidic device, in accordance with the present disclosure. More specifically, FIGS. 2A-2B illustrate a portion 235 of a microfluidic device, which may include at least some of substantially the same features and components as previously described by FIG. 1A. For example, the microfluidic devices of FIGS. 2A-2B include a reservoir 202, a cell-poration region 204, an optical-detection region 20, and a fluid ejector 212, but with the microfluidic devices including two of each of the cell-poration regions 204, optical-detection regions 208, and fluid ejectors 212 coupled to the reservoir 202. The common features and components are not repeated for ease of reference.

The example cell-poration regions 204 of FIGS. 2A-2B include a microfluidic channel 237 with a set of electrodes 206-1, 206-2 used to perform electroporation. In FIG. 2A, the cell-poration region 204 includes a constriction portion 207 which is attenuated from the remaining portions of the microfluidic channel 237. A first electrode 206-1 is disposed upstream of the constriction portion 207 and a second electrode 206-2 is disposed downstream of the constriction portion 207.

In FIG. 2B, the cell-poration region 204 includes a microfluidic channel 237 that is serpentine-shaped and includes a first electrode 206-1 and a second electrode 206-2 that extend through the microfluidic channel 237. More particularly, the first and second electrodes 206-1, 206-2 extend across a plurality of portions of the microfluidic channel 237, such that the cell crosses through the electric field provided by the first and second electrodes 206-1, 206-2 (and in response to a voltage or current applied to the electrodes 206-1, 206-2) a plurality of times as the cell flows though the microfluidic channel 237 and to the optical-detection region 208.

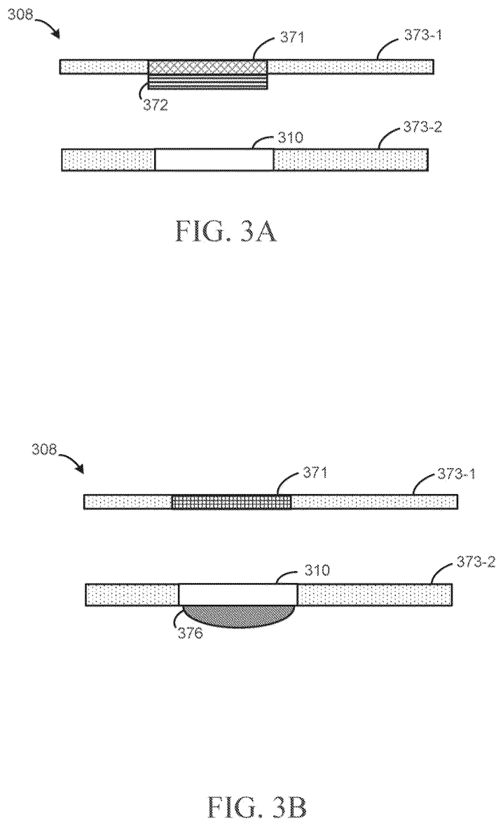

FIGS. 3A-3B illustrate example optical-detection regions of a microfluidic device, in accordance with the present disclosure. The optical-detection regions 308 illustrated by FIGS. 3A-3B may be implemented in any of the microfluidic devices and/or apparatuses as illustrated by FIGS. 1A-2B and FIGS. 4A-6D.

As previously described, the optical-detection region 308 includes an optically transparent window 310 associated with a wall 373-2 of the optical-detection region 308. In some examples, the wall 373-2 may be a substrate or in a substrate of the microfluidic device, such as in a side wall of the device.

In some examples, and referring to FIG. 3A, the optical-detection region 308 includes an opposing wall 373-1 that includes an optical detector 371 optically coupled to an optical filter 372. The optical detector 371 may be associated with the wall 373-1 which opposes the wall 373-2 having the optically transparent window 310 or may be located on a traverse wall relative to the optically transparent window 310. A traverse wall, as used herein, refers to or includes a wall that is located along a plane that transects a plane of the optically transparent window 310, e.g., is perpendicular. In some examples, the microfluidic device may include a light directing element which directs light towards the opposing wall 373-1. The optical detector 371 may include a band pass filter with a p-n junction diode, a camera detector, a charged coupling device (CCD) detector, a photo-sensor, a complementary metal oxide semiconductor (CMOS) detector, among other, which is located on the opposing wall 373-1.

In some examples, the optical detector 371 includes a photo-sensor, such as a photodetector or photoelectric sensor. The photo-sensor may include a light emitter and a receiver. The light emitter may emit light to be received by the receiver. In some examples, the photo-sensor is a photoelectric sensor that may have a through-beam arrangement where the emitter and the receiver are positioned on opposite sides of a flow way. In yet other examples, a photoelectric sensor may have a retroreflective arrangement where the emitter and the receiver may be positioned on the same side of a flow way and a reflector may reflect and/or direct light to the photoelectric sensor. An interruption in the light detected by the photoelectric sensor may evidence passage of a cell.

As shown by FIG. 3B, in some examples, the optically transparent window 310 forms a collimating lens 376 or has a collimating lens 376 attached thereto. The optical-detection region 308 may further include an optical detector 371, as previously described. The collimating lens 376 may narrow and focus light towards a specific direction. In some examples, the collimating lens 376 may filter out light rays which are not traveling parallel to the direction of light rays that pass through the collimating lens 376. In some examples, the collimating lens may be a refractive lens, a Fresnel lens, or a diffractive lens. A refractive lens bends light rays. A Fresnel lens is a specific type of composite compact lens having a large aperture and short focal length. Fresnel lenses may be thinner than other lenses. A diffractive lens has thin elements that may make use of the wave nature of light. Diffractive lenses modify the phase of light using micro-structure patterns fabricated on a surface of the lens. Light that passes through sunken areas travels faster than light that travels through higher areas of the lens creating controlled phase delay. In some examples, the collimating lens may be a lens that is refractive, brazed, saw-tooth, amplitude, binary, quaternary, or sinusoidal. A refractive lens may allow for about ninety-five % to about a hundred % collimation when fabricated using gray scale lithography and polishing. In some examples, a refractive lens may be a gradient refractive lens. A blazed lens may allow for about ninety % to about a hundred % collimation when fabricated using gray scale lithography and diamond turning. A saw-tooth lens may allow for about eighty-five % to about ninety % collimation when fabricated by diamond turning. An amplitude lens may allow for about eight % to about ten % collimation when fabricated using lithography. A binary lens may allow for about thirty-five % to about forty % collimation when fabricated using lithography. A quaternary lens may allow for about seventy % to about eighty-five % collimation when fabricated using lithography. A sinusoidal lens may allow for about twenty-five % to about thirty-five % collimation when fabricated using holographic exposure. The collimating lens 376 may direct light into optical-detection region 308.

In some examples, the opposing wall 373-2, which may be positioned transverse or opposite the optically transparent window 310 may include an optical detector 371. The term “defines” is inclusive of examples where the wall partially or fully defines the optical-detection region 308. A component attached to or embedded in a wall may likewise partially define the optical-detection region 308, e.g., recessed or partially recessed optical detector or other component. The optical detector 371 may be positioned to receive optical signals, such as fluorescent signals. In some examples, the optical detector 371 may include a pin-photodiode, an avalanche photodiode, a phototransistor, a multi-junction photodiode, a CCD, a CMOS device, a photo-sensor, an image sensor, a photo-resistor, a pyroelectric detector, a thermopile, a CMOS image sensor, a CMOS image sensor, and combinations thereof. In other examples, the optical detector 371 includes a pin-photodiode. In some examples, the optical detector 371 includes a multi-junction photodiode. In other examples, the optical detector 371 includes a camera sensor. In other examples, the optical detector 371 includes a CMOS image sensor. In some examples, the optical detector 371 includes a CCD image sensor. In some examples, the optical detector 371 is coupled to an optical filter, such as a band pass filter, such as illustrated by FIG. 3A. In various examples, the optical detector 371 may be an imaging or non-imaging optical detector.

However, examples are not limited to the optical-detection regions 308 illustrated in FIGS. 3A-3B. In some examples, external optical sensing circuitry may be used. Further, in some examples, a sensor may be disposed before the optical-detection region 308, such as an impedance sensor, to sense impedance of the fluid flowing to the optical-detection region 308, as previously described in connection with FIGS. 1D-1E.

The microfluidic device and portions thereof illustrated by FIGS. 1A-3B may include a plurality of components combined in ways not shown specifically in FIGS. 1A-3B, as any of the components shown may be combined for a given application. In some examples, the microfluidic device may include multiple reservoirs, one for each of the fluid containing the cells and the molecular probes. In some examples, the microfluidic device may include a single fluid reservoir with multiple cell-poration regions, optical-detection regions, and fluid ejectors fluidically coupled to the fluid reservoir. A microfluidic device with a plurality of components may allow for sorting of multiple cell types at the same time. When sorting multiple cell types, differing cell types in the fluid may be exposed to different molecular probes. For example, cell A may bind with molecular probe A and cell B may bind with molecular probe B, and the cell sorting used to handle and dispense both types of cells separately.

The microfluidic devices may be fabricated using integrated circuit microfabrication techniques, such as electroforming, laser ablation, anisotropic etching, sputtering, dry etching, wet etching, photolithography, casting, moulding, stamping, machining, spin coating, laminating, and the like. The microfluidic device may be manufactured from a variety of substrate materials. For example, the microfluidic device may include a material selected from glass, quartz, polyamide, polydimethylsiloxane, silicon, SU8, polystyrene, polycarbonate, polymethyl methacrylate, polyethylene, poly(ethylene glycol) diacrylate, polypropylene, perfluoroalkoxy, fluorinated ethylene propylene, polyurethane, cyclic olefin polymer, cyclic olefin copolymer, phenolics, or a combination thereof. In some examples, the microfluidic device is fabricated from polydimethylsiloxane. In some examples, the microfluidic device is fabricated from polycarbonate. In some examples, the microfluidic device is fabricated from silicon. In some examples, the microfluidic device is fabricated from SU8.

In various examples, the microfluidic devices described above may form part of an apparatus used to perform cell sorting. The apparatus may be used to sort cells to a substrate, such as single cell sorting. Examples are not limited to single cell sorting, and may include dispensing a population of cells to different portions of the substrate based on detection of a cellular membrane bound to or an intracellular target. As previously described, the intracellular target may be detected in response to measuring a fluorescent signal associated with binding between the molecular probe and the intracellular target.

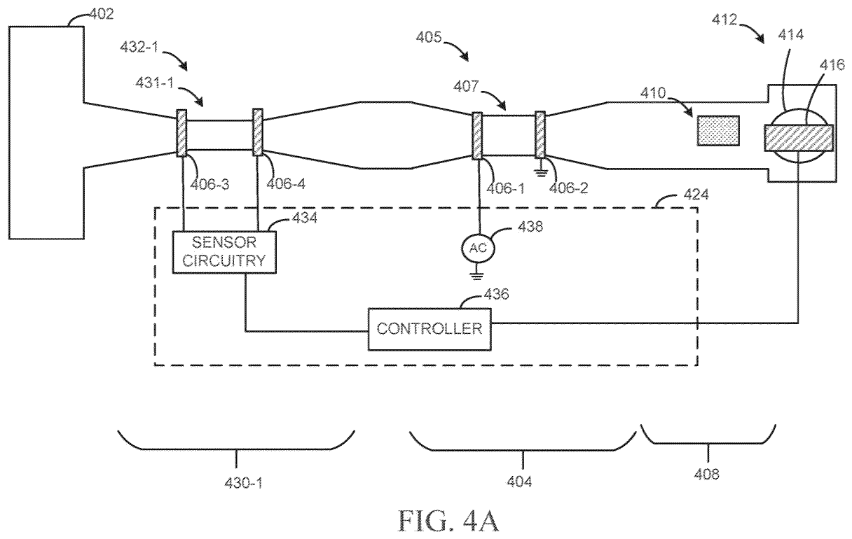

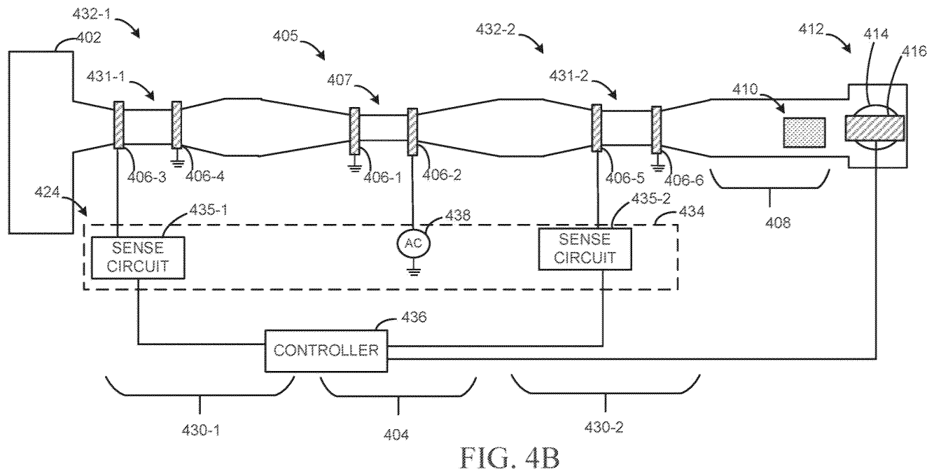

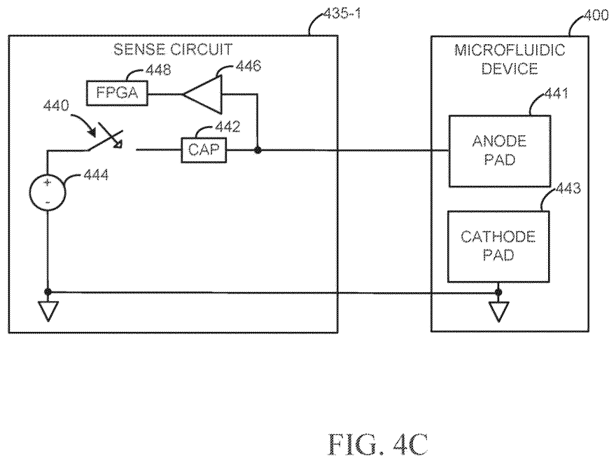

FIGS. 4A-4C illustrate example microfluidic devices and coupled circuitry, in accordance with the present disclosure. The microfluidic devices illustrated by FIGS. 4A-4B include substantially the same features and components as the microfluidic device 100 of FIGS. 1D-1E, with the additional circuitry illustrated and are numbered according. The common features and components are not repeated for ease of reference. For instance, each of the microfluidic devices of FIGS. 4A-4B include a reservoir 402, a cell-poration region 404, an optical-detection region 408, and a fluid ejector 412.

As previously described, the microfluidic devices may include a variety of variations, at least some of which are illustrated by FIGS. 4A-4B. For example, FIG. 4A illustrates an example microfluidic device that includes a first sensor region 430-1 including a first microfluidic channel 432-1 and a first sensor 431-1, as previously described by FIG. 1D. FIG. 4B illustrates an example microfluidic device that includes a first sensor region 430-1 including a first microfluidic channel 432-1 and a first sensor 431-1 and a second sensor region 430-2 including a second microfluidic channel 432-2 and a second sensor 431-2, as previously described by FIG. 1E.

In the examples illustrated by FIG. 4A-4B, circuitry 424 is coupled to respective components of the microfluidic devices. The circuitry 424 may include or form part of a fluid dispensing device. A fluid dispensing device may be an ink-jet based dispensing device that may dispense picoliters or nanoliters of fluid into specific locations on a substrate. The fluid dispensing device may use a microfluidic device for dispensing fluid. The microfluidic device may operate similar to a printhead. The fluid dispensing device may include a substrate transport assembly to move the substrate, as further described herein.

Referring to FIG. 4A, the circuitry 424 includes sensor circuitry 434, an alternating current (AC) source 438 coupled to an electrode 406-1 of the set of electrodes 406-1, 406-2, and a controller 436. The sensor circuitry 434 may couple to the first sensor 431-1 and obtain sensor signals, such as using a first sensing electrode 406-3 and a second sensing electrode 406-4. The sensor signals may include a sensor signal associated with a cell as the cell passes by the first sensor 431-1 in the first microfluidic channel 432-1. As further illustrated by and referring to FIG. 4B, the sensor circuitry 434 may include a first sense circuit 435-1 and a second sense circuit 435-2 respectively coupled to the first sensor 431-1 and the second sensor 431-2. The sensor signals may include a first sensor signal associated with a cell as the cell passes by the first sensor 431-1 in the first microfluidic channel 432-1 and a second sensor signal associated with the cell as the cell passes by the second sensor 431-2 in the second microfluidic channel 432-2.

The controller 436 is coupled to the sensor circuitry 434 and fluid ejector 412 to cause flow of fluid including a cell to the first sensor region 430-1 and through the cell-poration region 404 (which may include constriction portion 407), the second sensor region 430-2, and through the optical-detection region 408 including the optically transparent window 410 via actuation of the fluid ejector 412 coupled to the optical-detection region 408, and apply electric fields within the cell-poration region 404, the first sensor region 430-1 and/or the second sensor region 430-2 via the set of electrodes 406-1, 406-2, the first sensor 431-1, and/or the second sensor 431-2 to transfect or transform the cell and to sense whether or not cell transfection or transformation occurs and/or detect a cell approaching the cell-poration region 404 and/or optical-detection region 408, as previously described. The fluid ejector 412 may be actuated by the controller 436 sending or transmitting an electrical signal to the fluidic actuator 416 of the fluid ejector 412 to cause the fluidic actuator 416 to actuate and which causes the fluid to flow, as previously described. The controller 436 may apply the electric fields by transmitting electrical signals to the sensor circuitry 434 and/or the AC source 438, which causes the application of the electric fields within the cell-poration region 404, the first sensor region 430-1, and/or the second sensor region 430-2 via the cell-poration mechanism 405, the first sensor 431-1, and/or the second sensor 431-2.

As shown by the cell-poration mechanism 405 of FIG. 4A, an electrode 406-2 of the pair of electrodes 406-2, 406-2 is grounded and the other electrode 406-1 is coupled to the AC source 438. A potential is applied across the electrodes 406-1, 406-2 by applying an electrical signal to the electrode 406-2 via the AC source 438, which causes the electric field within the cell-poration region 404.

In some examples, and referring to FIGS. 4A-4B, the first sensor 431-1 and/or the second sensor 431-2 include a pair of sensing electrodes 406-3, 406-4, 406-5, 406-6 with one electrode 406-4,406-6 of the pair being grounded and the other electrode 406-3, 406-5 of the pair coupled to a component of the sensor circuitry 434, such as to sense circuits 435-1 and/or 435-2. The pair of sensing electrodes 406-3, 406-4, 406-5, 406-6 provide an electric field there between. Fluid containing the cell is conductive, as previously described. As the cell flows through the electric field, an impedance measure is obtained as a sensor signal by the first sensor 431-1 and/or the second sensor 431-2. For example, the sensor circuitry 434 applies a voltage or current to the one sensing electrode 406-3, 406-5 of the pair, with the other sensing electrode 406-4, 406-6 being grounded, and which causes the electric field to be applied within the first microfluidic channel 432-1 or the second microfluidic channel 432-2. The resulting impedance measure may be indicative of a cell property, such as a size of the cell, cell viability (e.g., alive or dead), single cells or clumps of cells or debris, among other properties. The impedance measure may be used to trigger activation of cell poration and/or of coupled optical sensing device. For example, the controller 436 may determine that the fluid contains a clump of cells or dead cells, and may eject the fluid without applying an electrical field for electroporation, with increased fluid flow through the cell-poration region 404, and/or without obtaining a measured optical signal from the optical-detection region 408. In the example illustrated by FIG. 4B, the sensor circuitry 434 includes sense circuits 435-1, 435-2 coupled to the first sensor 431-1 and second sensor 431-2 of the microfluidic device. Although the above describes impedance measures or signals, examples are not limited to impedance sensors and may include other types of signals which obtain other electrical signals.

Although not illustrated by FIGS. 4A-4B, the controller 436 may further communicatively couple to a light source and optical detector. The light source and optical detector may form part of the optical-detection region 108, such as illustrated by FIGS. 3A-3B, and/or may form part of an optical sensing device that is separate from the microfluidic device. The controller 436 may instruct the light source or the optical sensing device to emit excitation light toward the optically transparent window 410 and may receive the measured emitted light, in response to the excitation light, from the optical detector. The excitation light may excite the molecular probe, if bound to the intracellular target, and in response, the molecular probe may emit light that includes a fluorescent signal associated with the molecular probe being bound to the intracellular target. That is, the molecular probe may provide the particular fluorescent signal in response to binding to the intracellular target. Prior to binding, the particular fluorescent signal is not present.

Based on the detection of the particular fluorescent signal (or not), the controller 436 causes the fluid ejector 412 to eject the cell from the microfluidic device to a select region of a substrate based on the determined cell property. The region may be selected based on the cell property, such as dispensing dead cells or clumps of cells and/or debris to a waste region, single cells or other cells of a particular type to a select region or a group of regions, among other variations. The cell may be ejected by ejecting a volume of the fluid containing the cell via the fluid ejector 412 with the fluidic actuator 416 and the coupled nozzle 414, as previously described.

In some examples, the fluid includes a plurality of cells or other molecules. The controller 436 may determine cell properties of the plurality of cells, cause the fluid ejector 412 to eject each of the plurality of cells from the microfluidic device to select regions of the substrate based on the determined cell properties, and store a dispense map indicative of the select regions of the substrate that the plurality of cells are ejected to and as associated with the respective cell properties of the plurality of cells.

A dispense map, as used herein, includes and/or refers to data identifying properties of cell(s) within regions or classifying cells within the regions, e.g., wells, of a substrate. For example, the region may be classified as including a particular type of cell (e.g., cell or not, dead or alive, including the intracellular target or note) and/or a target number of cells or a single cells. The dispense map may identify regions of the substrate with target cell populations. The impedance signals or other electrical signals from the first and/or second sensors 431-1, 431-2, along with the optical signal from the optical detector, may be used to identify the cell properties, such as the type of cell, the size of the cell, identify clusters or clumps of cells, debris, and/or signal noise. Based on the determined properties, the dispense map may be generated by indicating which region of the substrate includes target cells, a target number of cells, and/or waste. In some examples, the map may be generated in real time and/or on-the-fly while the microfluidic device and/or controller 436 is continuing to dispense fluid into further regions of the substrate. As used herein, real time refers to or includes processing of signals or other data within a threshold amount of time, e.g., seconds or milliseconds. On-the-fly, as used herein, refers to or includes processing that occurs while the fluid is in motion and/or another process is in progress.

A target cell population, as used herein, refers to or includes a defined number of cells, or n-cells, of a particular type to be dispensed into a region of a substrate. A target cell population may include a single cell or multiple cells which exhibit the intracellular target. In some examples, the target cell population includes a single cell and/or a specific type of cell which may be identified by the properties detected. However, examples are not so limited. In various examples, different regions of the substrate may have different target cell populations. The region refers to or includes a particular location of a substrate to which a cell or a cell population is to be dispensed. The region may be a particular well on a micro-well plate or other types of substrates.

The controller 436 may include a processor and memory. Memory may include a computer-readable storage medium storing a set of instructions. Computer-readable storage medium may include Read-Only Memory (ROM), Random-Access Memory (RAM), Electrically Erasable Programmable Read-Only Memory (EEPROM), flash memory, a solid state drive, physical fuses and e-fuses, and/or discrete data register sets. In some examples, computer-readable storage medium may be a non-transitory storage medium, where the term “non-transitory” does not encompass transitory propagating signals.

The processor may be a central processing unit (CPU), a semiconductor-based microprocessor, a graphics processing unit (GPU), a microcontroller, special purpose logic hardware controlled by microcode or other hardware devices suitable for retrieval and execution of instructions stored in the non-transitory computer-readable storage medium, or combinations thereof. The controller 436 may fetch, decode, and execute instructions, as described herein. As an alternative or in addition to retrieving and executing instructions, the controller may include at least one integrated circuit (IC), other control logic, other electronic circuits, or combinations thereof that include a number of electronic components for performing the functionality of instructions.

FIG. 4C illustrates a close-up view of an example sense circuit 435-1 of the sensor circuitry 434 of FIGS. 4A-4B. Referring to FIG. 4A, the sensor circuitry 434 may be implemented as illustrated by FIG. 4C. Referring to FIG. 4B, each of the sense circuits 435-1, 435-2 may be implemented as illustrated by FIG. 4C. Referring back to FIG. 4C, the sense circuit 435-1 includes a capacitor 442, a power source 444, a switch 440, a ground path, an analog-to-digital (A/D) circuit 446, and a field programming gate array (FPGA) 448 or other processing circuitry. When the switch 440 is in a closed position, the power source 444 charges the capacitor 442 which is coupled to an anode pad 441 of a first sensing electrode of the first sensor (or the second sensor) of the microfluidic device 400 and causes an electric field within the first (or second) microfluidic channel of the microfluidic device 400. The capacitor 442 may be charged for a first period of time, and then the switch 440 is transitioned to an open position, and a measurement is obtained for a second period of time. The first and second periods of time may be at fixed intervals to obtain a plurality of sensor signals associated with a plurality of cells. The ground path is coupled to the cathode pad 443 of a second sensing electrode of the first sensor (or second sensor). In some examples, the cathode pad 443 is shared between the first sensor, the second sensor, and/or the set of electrodes of the cell-poration mechanism. The discharge of the capacitor 442 is observed using the A/D circuit 446 and FPGA 448. For example, in response to the cell or clump of cells passing by, the capacitor 442 discharges, which is captured by and converted to a digital signal by the A/D circuit 446 and identified by the FPGA 448. The FPGA 448 may be in communication with or form part of the controller.

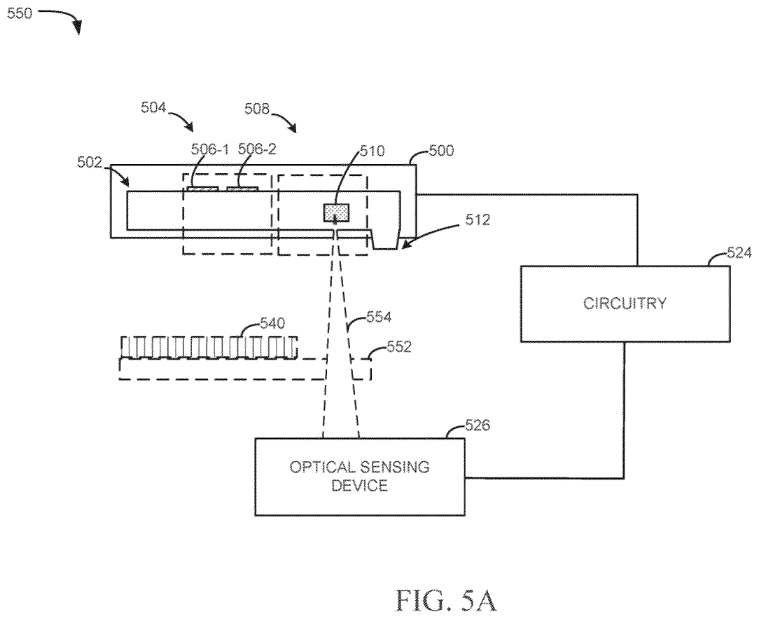

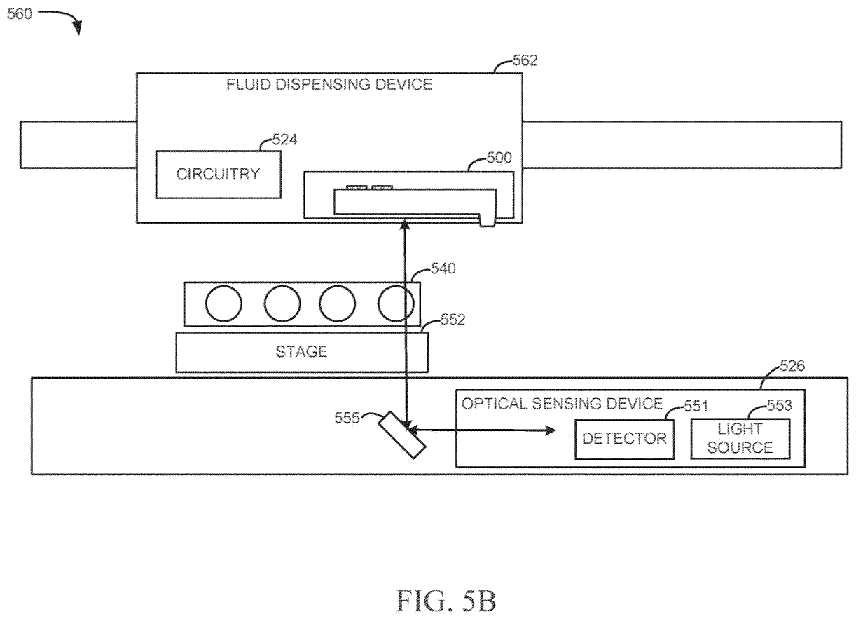

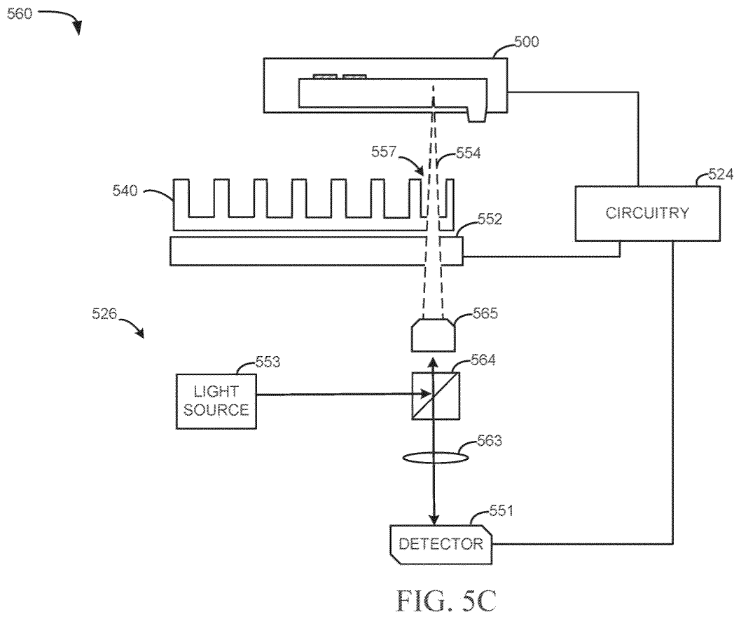

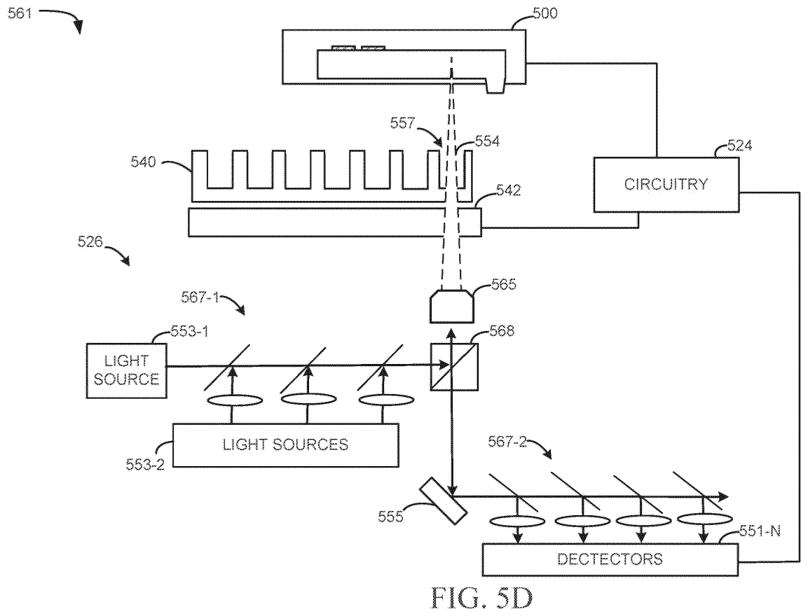

FIGS. 5A-5D illustrate different example apparatuses including a microfluidic device, optical sensing device, and circuitry, in accordance with the present disclosure. Each of the apparatuses 550, 560, 561 illustrated by FIGS. 5A-5D may include a microfluidic device illustrated by any of FIGS. 1A-4B or circuitry illustrated by FIGS. 4A-4C. More particularly, the microfluidic devices 500 of FIGS. 5A-5D may comprise an example implementation of, or comprise at least some of substantially the same features and components as any one of the example microfluidic devices in association with any of FIGS. 1A-4C, the common features and components are not repeated for ease of reference.

As shown by FIG. 5A, an example apparatus 550 includes a microfluidic device 500, an optical sensing device 526, and circuitry 524. The microfluidic device 500 includes a reservoir 502 to store a fluid containing at least a cell and at least a molecular probe, a cell-poration region 504 fluidically coupled to the reservoir 502 and including a set of electrodes 506-1, 506-2, an optical-detection region 508 fluidically coupled to the cell-poration region 504 and including an optically transparent window 510 associated with a wall of the optical-detection region 508, and a fluid ejector 512 fluidically coupled to the optical-detection region 508.

The optical sensing device 526 is to measure emitted light responsive to excitation light provided toward the optical-detection region 508. For example, the optical sensing device 526 may include a light source 553 and optical detector 551, as illustrated by FIG. 5B. Referring back to FIG. 5A, the light source may provide excitation light 554 toward the optically transparent window 510 of the optical-detection region 508 and/or the optical detector measures emitted light in response to the excitation light. The light source may be positioned to emit or reflect light toward the optically transparent window 510. In In some examples, the optical sensing device 526 may include the light source to provide excitation light 554 and the optical detector may form part of the microfluidic device 500, as previously described in connection with FIGS. 3A-3B. The optical detector may measure optical signals emitted from components within the fluid present in the optical-detection region 508, such as the fluorescent signal associated with the molecular probe binding to the intracellular target.

The circuitry 524 is communicatively coupled to the microfluidic device 500 and the optical sensing device 526 to drive flow of the fluid through the microfluidic device 500 via the fluid ejector 512 and to detect binding of the molecular probe to an intracellular target in the cell via the measured emitted light. In some examples, the circuitry 524 is separate from the microfluidic device 500 and in other examples, is an integrated part. The circuitry 524 may include the electrical connections to the fluidic actuator, the sensor(s), and/or the electrodes of the cell-poration mechanism. The circuitry 524 may include a controller to control timing the fluid flow through the microfluidic device, control electric fields and/or shear force, cause ejection of fluid from the microfluidic device 500 through the ejection nozzle, cause movement of the microfluidic device 500 and/or the stage 552 (as described below) align individual regions of a substrate 540, such as a multi-well plate with the nozzle of the fluid ejector 512, or set a vertical distance between fluid in the substrate 540 and the nozzle.

As described above, in some examples, the circuitry 524 receives feedback from sensors, including the optical detector, which may indicate whether or not target cell or other particle in the fluid has passed through the sensor in the microfluidic device 500. Depending on the sensor and measured data, the feedback may indicate whether or not perform cell poration, poration parameters, whether or not to perform optical detection, and/or an alignment of which region of the substrate 540 a fluid droplet may be ejected into. When feedback received indicates that a target cell is present, then the circuitry 524 directs alignment of an empty region (e.g., well) in the substrate 540 (e.g., multi-well plate) with the nozzle of the microfluidic device 500. Following the alignment, the circuitry 524 may direct the fluid ejector 512 to eject a droplet of the fluid with the target cell therein into the empty region, thereby allowing individual regions of the substrate 540 to be filled with an individual target cell. When feedback received indicates that a target cell is not present, then the circuitry 524 may direct alignment of a waste region of the substrate 540 with the nozzle of the fluid ejector 512. Following alignment of the waste region and the nozzle, the circuitry 524 may direct the fluid ejector 512 to eject a fluid droplet of the fluid into the waste region which includes fluid droplets not of interest for further processing and/or assessment.

In some examples, as shown by FIG. 5A, the apparatus 550 may further comprise a substrate 540 and a stage 552 coupled to the substrate 540. The substrate 540 may include a plurality of regions, such as a multi-well plate having a plurality of wells to eject fluids to.

In some examples, the circuitry 524 is communicatively coupled to the stage 552 to instruct the stage 552 to move the substrate 540 relative to the fluid ejector 512, such that the fluid ejector 512 is aligned with a select region of the plurality of regions of the substrate 540. The fluid ejector 512 may selectively eject a fluid droplet of the fluid containing the cell from the microfluidic device 500 to the select region of the plurality of regions of the substrate, which may be based on the detecting binding of the molecular probe to the intracellular target or not, and/or sensor signals, as previously described.

In some examples, the fluid may include a plurality of cells and a plurality of the molecular probe. The circuitry 524 may assess for a presence of the intracellular target in the plurality of cells via the measured emitted light associated with each of the plurality of cells, and actuate the fluid ejector 512 to cause selective ejection of the plurality of cells from the microfluidic device 500 to a plurality of regions of the substrate 540 and based on the assessed presence. To selectively eject the plurality of cells to different regions of the substrate 540, the circuitry 524 may instruct the stage 542 to move the substrate 540 relative to the fluid ejector 512, as described above.

FIG. 5B illustrates an example implementation of the apparatus 560 of FIG. 5A, with additional and optional component illustrated including a fluid dispensing device 562. The common features and components are not repeated for ease of reference.

As shown by FIG. 5B, the apparatus 560 may include a fluid dispensing device 562. The fluid dispensing device 562 includes a substrate transport assembly and the circuitry 524. The substrate transport assembly may include a stage 552 coupled to one of the substrate 540 and the fluid dispensing device 562 to move a position of the substrate 540 with respect to the microfluidic device 500. The fluid dispensing device 562 may include additionally non-illustrated components, such as a mounting assembly and a power supply that provides power to the various electrical components of the fluid dispensing device 562 and the microfluidic device 500 mounted therein. The fluid dispensing device 562 may control a fluid ejector of the microfluidic device 500 to dispense droplets of fluid to the substrate 540. The fluid dispensing device 562 may cause flow of a fluid from a reservoir, through a cell-poration region and an optical-detection region, and to the fluid ejector, and then cause the fluid ejector to eject a volume of the fluid from the fluid ejection device to a region of the substrate 540.

As described above, the apparatus 560 may include the substrate 540. The substrate 540 may include different regions, such as wells of a well plate, with each region getting a cell or a cell population depending on determined properties. These dispense locations may be specific target regions on the substrate surface, such as cavities, microwells, channels, indentation into the substrate, or other regions of the substrate.

In the example illustrated by FIG. 5B, the optical sensing device 526 includes an optical detector 551, a light source 553, and a mirror 555. The light source 553 is positioned such that excitation light may be emitted or reflected toward the optically transparent window in the microfluidic device 500.

A variety of different light sources may be used, such as a laser, a light-emitted diode (LED), an infrared light source, a near infrared light source, xenon arc lamp, mercury light lamp, halogen light lamp, among other light sources. The light source 553 may emit the excitation light at a particular electromagnetic energy to excite the molecular probe bound to the intracellular target. In some examples, the light source 553 may use bright field imaging to illuminate the fluid. Bright field imaging may allow for some cells in the fluid to appear dark when surrounded by the bright viewing field. In other examples, as further illustrated herein, the light source may include a multi-spectral light source.

The wavelength range of the excitation light, sometimes herein referred to as an emission range, may be correlated to the fluorescent signal of the molecular probe when bound to the intracellular target. For example, the light source 553 may emit excitation light in a wavelength range that overlaps with the wavelength of the fluorescent signal. In some examples, the correlation may occur by the light source 553 which includes a plurality of individual illuminators that may be selectively turned on and off. In some examples, as further illustrated herein, excitation light emitted by the light source 553 may be emitted towards a filter cube, a spectral filter, a beam splitter, a reflective direction source, such as the mirror 555, a prism, and combinations thereof. In some examples, wavelengths of excitation light that do not correlate with the excitement energy of the molecular probe may be filtered or directed away from the optically transparent window. Further, the excitation light may be directed or focused using the mirror 555 and/or an objective lens.

The optical detector 551 may measure or receive and pass along the optical measure. In some examples, the optical detector 551 may include a fluorimeter, a photoluminescence spectrometer, a semiconductor such as a p-n junction diode, a photodiode, a phototransistor, or a combination thereof. In yet other examples, the optical detector 551 may be a detector array. The optical detector 551 may be positioned to detect fluorescence from a cell bound to the molecular probe. The optical detector 551 may be a standalone component and/or may be integrated with the microfluidic device 500, as described above. In some examples, the optical detector 551 may be positioned on a linear substrate designed to be placed between a stage 552 and the substrate 540.

In some examples, the excitation light may be provided through the stage 552 and the substrate 540, such as through a waste region, with both the stage 552 and the substrate 540 including optically transparent windows. For example, the stage 552 and substrate 540, or portions thereof, may include or be optically transparent to allow for positioning of the light source 553 and/or the optical detector 551 below the stage 552. As described above, the stage 552 and the microfluidic device 500 are movable in relation to one another. The movable component may be the stage 552, the microfluidic device 500, or a combination thereof. The movement may allow for the aligning of individual regions of the substrate 540, such as wells of a multi-well plate, with the nozzle of the fluid ejector on the microfluidic device 500. The movement may also align a height of the substrate 540 with respect to the bottom of the nozzle of the fluid ejector. In some examples, the substrate 540 and the nozzle may be spaced apart at from 0.5 mm 5 to 5 mm, from 1 mm to 4 mm, or from 1 mm to 3 mm. Minimizing a distance between the top of the substrate 540 and the nozzle may, in some instances, minimize fluid loss from a misdirected or displaced droplet, e.g., being blown off course, during ejecting and depositing in a region of the substrate 540. In some examples, the stage 552 may be modified to permit movement of the light source 553, the optical detector 551, or a combination thereof, a corollary amount when the stage 552 is moved. In some examples, the stage 552 may include a coupling for mounting of the light source 553, the optical detector 551, or a combination thereof.

FIG. 5C illustrates an example implementation of the apparatus 560 illustrated by FIG. 5B, with additional optical components illustrated. The common features and components are not repeated for ease of reference.

As shown by FIG. 5C, the optical sensing device 526 includes a light source 553 and optical detector 551, as previously described. The optical sensing device 526 further includes a filter cube 564, an objective 565, and a tube lens 563. The light source 553 emits the excitation light 554 toward the filter cube 564. The filter cube 564 selectively passes excitation light 554 of select wavelengths to excite the fluorophore associated with the molecular probe binding to the intracellular target toward and through the stage 552 and a region 557 of the substrate 540 and toward an optically transparent window of the microfluidic device 500. The objective 565 may be disposed between the filter cube 564 and the stage 552 that passes and/or focuses the excitation light toward the optically transparent window. The region 557 may include a waste region or regions (e.g., at least one junk well). In response to the presence of the fluorophore, e.g., the molecular probe bound to the intracellular target, the fluorophore is excited by the excitation light 554 and emits a fluorescent signal toward the filter cube 564, which passes the emitted light of select wavelengths through the tube lens 563 and toward the optical detector 551, while reflecting other wavelengths, such as backscattered light, away from the optical detector 551.

In an example operation, the cell passes through the cell-poration region and the molecular probe is introduced within the cell. The molecular probe binds to the intracellular target, such as specific mRNA inside the cell, and becomes fluorescent if the intracellular target is present. The fluid ejector is positioned over the particular region 557 and fluid is dispensed into the region 557 until the cell with the intracellular target is observed. The dispensing is then stopped and the stage 552 is moved such that the fluid ejector 512 lines up with a different region of the substrate 540, and fluid including the cell is dispensed to the region. The fluid ejector 512 is returned to be over the particular region 557 and the process in repeated.

Different modes of operation may be used. An example mode of operation includes detecting the cell with a sensor, such as an impedance sensor, and in response, turn on the electroporation electrodes to transfect or transform the cell, and then fluorescently interrogate the cell. In another mode of operation, once the cell is sensed with the sensing electrodes of the impedance sensor, the fluid ejector 512 is moved over the region 557 through which the optical-detection region is imaged to detect the cell. Once the cell is detected by fluorescence, the fluid ejector 512 moves once again and dispenses again, and then moves back to the region 557. In some examples, the region 557 is large enough so that the meniscus height does not change appreciably during dispense, and therefore no extra focusing is used.

FIG. 5D illustrates another example apparatus 561. The apparatus 561 may comprise an example implementation of, or comprise at least some of substantially the same features and components as any of the apparatuses illustrated by FIGS. 5A-5C, but with the optical sensing device 526 including a multi-spectral optical sensing device. For example, the apparatus 561 includes a microfluidic device 500, a stage 552, a substrate 540, circuitry 524, and the optical sensing device 526, the common features and components are not repeated for ease of reference.

The example optical sensing device 526 includes a plurality of light sources 553-1, 553-2, first and second sets of spectral filters 567-1, 567-2, a beam splitter 568, an objective 565, a mirror 555, and a plurality of optical detectors 551-N. The plurality of light sources 553-1, 553-2 may selectively output excitation light of different wavelength ranges toward the first set of spectral filters 567-1 and to the beams splitter 568. A beam splitter includes an optical device that splits the excitation light in two light beams, with a first light beam being provided toward the objective 565 and to the microfluidic device 500 as excitation light and the second light beam being provided toward the optical detectors 551-N via the mirror 555. The second light beam is filtered by the second set of spectral filters 567-2 and provided to the optical detectors 551-N to use for comparing to the returned emitted light. The emitted light is provided to the optical detectors 551-N, which include different optical detectors or channels for each of the plurality of wavelength ranges.

By using a multi-spectral optical sensing device, a plurality of different molecular probes may be introduced within or into a cell concurrently, with each of the different molecular probes being associated with a different and optically distinguishable fluorescent signal, such as fluorescent signals that do not overlap in wavelength range. In the example illustrated by FIG. 5D, four different wavelength ranges are used which may allow for four different molecular probes which are associated with a different fluorescent signal. Examples are not so limited and may include between four to twenty or more different wavelength ranges detected and associated with four to twenty or more different molecular probes. Using multiple different molecular probes may allow for targeting multiple intracellular targets concurrently, such as multiple mRNAs of interest. The different molecular probes may be observed directly using the optical detectors 551-N. Further, the cells may be gated based on observations in several multiplexed channels and sorted, each multiplexed channel having a cell-poration region, an optical-detection region, and a coupled fluid ejector. For example, cells displaying the presence of a first intracellular target and third intracellular target may be placed in a first region of the substrate 540, and cells displaying a presence of a second intracellular target without the first intracellular target, without a third intracellular target, and without a fourth intracellular target may be placed in a second region of the substrate 540.

Various examples may include different variations to the cell-poration region, such as different ways to adjust the poration parameters. For example, microfluidic devices may include multiple fluid ejectors coupled to the microfluidic channel.

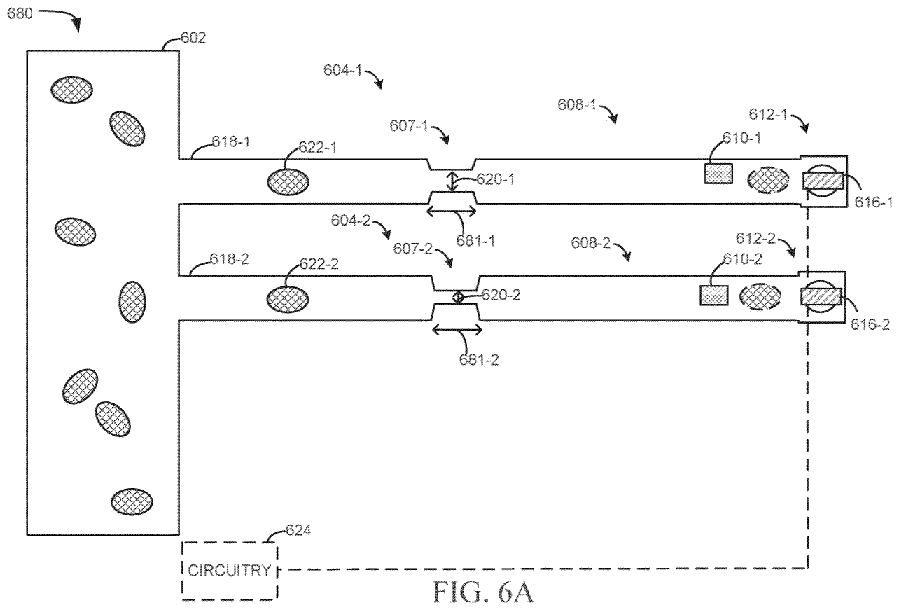

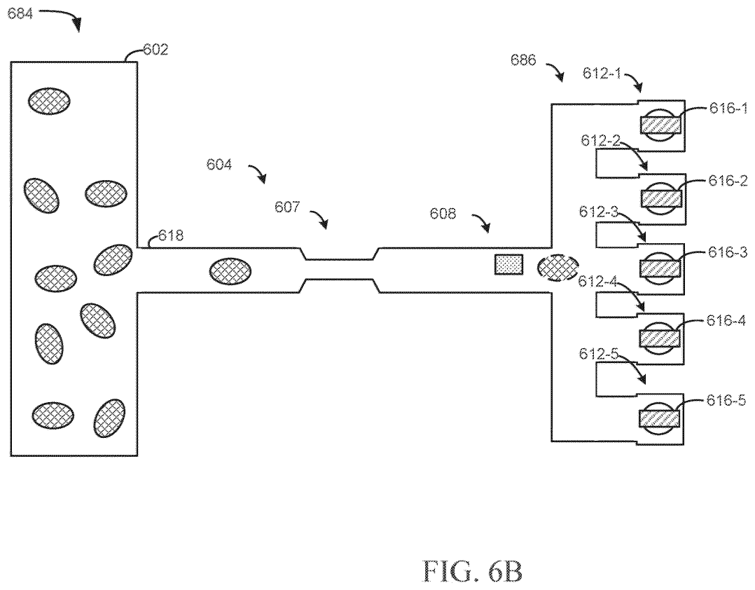

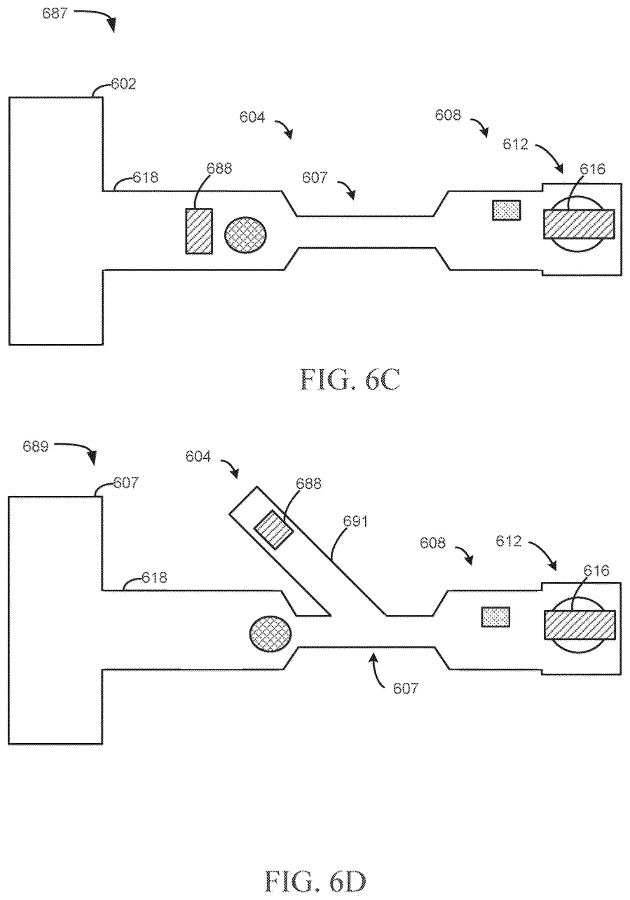

FIGS. 6A-6D illustrate further example microfluidic devices with a cell-poration region, optical-detection region, and fluid ejector, in accordance with the present disclosure. FIGS. 6A-6D illustrate an example of a microfluidic device, which may include at least some of substantially the same features and components as previously described by FIGS. 1A-1E. For example, the microfluidic device of FIGS. 6A-6D include a reservoir 602, a cell-poration region 604 (or regions 604-1, 604-2), and at least one fluid ejector 612-1, with some variations. The common features and components are not repeated for ease of reference.

FIG. 6A illustrates a microfluidic device 680 that includes multiple microfluidic channels 618-1, 618-2. The microfluidic device 680 may be used to identify and/or optimize mechanical poration parameters, such as shear forces and/or flow rates to use. The reservoir 602 is coupled to each of the first and second microfluidic channels 618-1, 618-2. Each of the first and second microfluidic channels 618-1, 618-2 include a cell-poration region 604-1, 604-2, an optical-detection region 608-1, 608-2 with an optically transparent window 610-1, 610-2, and a coupled fluid ejectors 612-1, 612-2.

The cell-poration regions 604-1, 604-2 may include different sized constriction portions 607-1, 607-2. For example, the first microfluidic channel 618-1 includes a first constriction portion 607-1 that includes a first circumference 620-1 that is attenuated from remaining portions of the first microfluidic channel 618-1. The second microfluidic channel 618-2 includes a second constriction portion 607-2 that includes a second circumference 620-2 that is attenuated from remaining portions of the second microfluidic channel 618-2.

The first constriction portion 607-1 and second constriction portion 607-2 include different dimensions from one another. In some examples, the different dimensions of the first constriction portion 607-1 and second constriction portion 607-2 include circumferences, lengths, and/or number of constriction sub-regions. For example, the first constriction portion 607-1 and second constriction portion 607-2 may have different circumferences 620-1, 620-2 from one another. In some examples, the first constriction portion 607-1 and second constriction portion 607-2 may have different lengths 681-1, 681-2 and different circumferences 620-1, 620-2 from one another.

The different dimensions may be used to provide different amounts of shear forces on different cells. In some examples, additional forces, such as different tensile and/or compression forces are applied on the cells. For example, the first constriction portion 607-1 includes the first circumference 620-1 and a first length 681-1, and the second constriction portion 607-2 includes the second circumference 620-2 and a second length 681-2. The second circumference 620-2 is smaller than the first circumference 620-1, and the second and first lengths 681-1, 681-2 are the same, in the particular example illustrated by FIG. 6A. However, examples are not so limited. In some examples, the circumferences 620-1, 620-2 may be the same and the lengths 681-1, 681-2 are different. In some examples, both the circumferences 620-1, 620-2 and the lengths 681-1, 681-2 are different. In some examples, the microfluidic device 680 may include more than two microfluidic channels with constriction portions, each having different dimensions. In some examples, the constriction portions 607-1, 607-2 may have different circumferences in a range of four to ten micrometer (μm) wide and different lengths in the range of eight to thirty μm long. The circumferences may be adjusted by changing the width or height of the respective microfluidic channels. In some examples, the first constriction portion 607-1 and second constriction portion 607-2 may have different numbers of constriction sub-regions.

The fluid ejectors 612-1, 612-2 are respectively in fluidic communication with the first microfluidic channel 618-1 and the second microfluidic channel 618-2 to cause movement of respective portions of the fluid from the reservoir 602 to the first microfluidic channel 618-1 and to the second microfluidic channel 618-2 and respectively through the first constriction portion 607-1 and the second constriction portion 607-2. Various fluid ejectors 612-1, 612-2 may be used to actively move fluid through the first and second microfluidic channel 618-1, 618-2 (and other microfluidic channels). The fluid ejectors 612-1, 612-2 may form part of the microfluidic device, and are controlled by circuitry 624.

In some examples, the circuitry 624 may control components of the microfluidic device 680. In some examples, the circuitry 624 forms part of the microfluidic device or is separate from the microfluidic device 680. The circuitry 624 may actuate the first fluid ejector 612-1 to direct a flow of portions of the fluid from the reservoir 602 into the first microfluidic channel 618-1 and through the first constriction portion 607-1, and may actuate the second fluid ejector 612-2 to direct a flow of portions of the fluid from the reservoir 602 into the second microfluidic channel 618-2 and through the second constriction portion 607-2 at different flow rates. In some examples, the different flow rates may be due to the different circumferences 620-1, 620-2 of the constriction portions 607-1, 607-2. In other examples and/or in addition, the different flow rates are due to actuating the first fluidic actuator 616-1 and second fluidic actuator 616-2 differently (e.g., for different amounts of time). In some examples, the circuitry 624 may store a list that identifies the set flow rate for the particular type of cell and which is different for the constriction portions 607-1, 607-2 and/or may generate the list, as previously described.

As an example, the circuitry 624 may be communicatively coupled to the first and second fluid ejectors 612-1, 612-2 to actuate the fluidic actuators 616-1, 616-2 to direct a flow of a first portion of the fluid from the reservoir 602 into the first microfluidic channel 618-1 and through the first constriction portion 607-1, and to direct a flow of a second portion of the fluid from the reservoir 602 into the second microfluidic channel 618-2 and through the second constriction portion 607-2. The first portion of the fluid may include a first cell 622-1 of the plurality of cells and the second portion may include a second cell 622-2 of the plurality of cells. In some examples, the circuitry 624 may actuate the fluidic actuator 616-1 to apply a first shear force on the first cell 622-1 in response to the flow through the first constriction portion 607-1, and thereby cause formation of apertures in the cell membrane of the first cell 622-1. The circuitry 624 may actuate the second fluidic actuator 616-2 to apply a second shear force on the second cell 622-2 in response to the flow through the second constriction portion 607-2, and thereby cause formation of apertures in the cell membrane of the second cell 622-2. In some examples, the circuitry 624 may actuate the fluidic actuators 616-1, 616-2 to apply sets of forces. For example, the circuitry 624 may apply a first set of compression, tensile, and shear forces, including the first shear force, on the first cell 622-1. The circuitry 624 may apply a second set of compression, tensile, and shear forces, including the second shear force, on the second cell 622-2. The circuitry 624 may further actuate the fluidic actuators 616-1, 616-2 to expose the first cell 622-1 and the second cell 622-2 to the molecular probe and to introduce the molecule probe within the first and second cells 622-1, 622-2, e.g., transfect or transform cells.

As described above, the different dimensions of the first constriction portion 607-1 and second constriction portion 607-2 may include a dimension selected from circumference, length, number of constriction sub-regions, and combinations thereof. In some examples, the circuitry 624 is to actuate the fluidic actuators to apply a first shear force on the first cell 622-1 in response to the flow through the first constriction portion 607-1, and thereby cause formation of apertures in the cell membrane of the first cell 622-1, apply a second shear force on the second cell 622-2 in response to the flow through the second constriction portion 607-2, and thereby cause formation of apertures in the cell membrane of the second cell 622-2, and expose the first and second cells 622-1, 622-2 to molecular probes to transfect or transform the first and second cells 622-1, 622-2. For example, the first fluidic actuator 616-1 may be actuated to cause the flow of fluid through the first microfluidic channel 618-1 and the second fluidic actuator 616-2 may be actuated to cause the flow of fluid through the second microfluidic channel 618-2, and which may occur simultaneously or at different times.

FIG. 6B illustrates a microfluidic device 684 that includes a microfluidic channel 618 fluidically coupled to a plurality of fluid ejectors 612-1, 612-2, 612-3, 612-4, 612-5. The plurality of fluid ejectors 612-1, 612-2, 612-3, 612-4, 612-5 may each include an ejection chamber with an ejection nozzle and fluidic actuators 616-1, 616-2, 616-3, 616-4, 616-5, which are selectively actuated to control a flow rate through the microfluidic channel 618. For example, circuitry may actuate one, two, three, four, or five of the fluidic actuators 616-1, 616-2, 616-3, 616-4, 616-5 at the same time to vary the flow rate and, therefore, vary the shear force applied on cells. In some examples, the fluid ejectors 612-1, 612-2, 612-3, 612-4, 612-5 may be fluidically coupled to the microfluidic channel 618 by a channel arrangement 686 which includes a first channel that intersects the microfluidic channel 618 and a plurality of branching channels in fluidic communication with the first channel and in which the fluid ejectors 612-1, 612-2, 612-3, 612-4, 612-5 are respectively disposed within. While the example of FIG. 6B illustrates five fluid ejectors, examples are not so limited and may include more or less fluid ejectors.

FIG. 6C illustrates an example microfluidic device 687 that includes another fluidic actuator 688 upstream of the cell-poration region 604. The fluidic actuator 688 is disposed with the microfluidic channel 618 and upstream of the constriction portion 607, and may include any of the previously described fluidic actuator as described in associated with the fluid ejector 612, such as a resistor. The other fluidic actuator 688 may be fired or pulsed to cause a vapor bubble within the microfluidic channel 618 to drive the fluid flow through the constriction portion 607 and/or cause shear forces on the cell. The flow rate may be dependent on a firing or pulse rate of fluidic actuator 616 and/or other fluidic actuator 688, which may be adjusted to set the flow rate and set the shear force applied to the cell. Circuitry, as previously described, may actuate the fluidic actuator 616 and/or other fluidic actuator 688 to direct the flow of the cell from the reservoir 602 into the microfluidic channel 618 and through the constriction portion 607 to the optical-detection region 608.

FIG. 6D illustrates an example microfluidic device 689, which is similar to the microfluidic device 687 of FIG. 6D, but with a branching microfluidic channel 691 that is coupled to the constriction portion 607. In some examples, the other fluidic actuator 688 is disposed with the branching microfluidic channel 691 that intersects the microfluidic channel 618 at the constriction portion 607. The other fluidic actuator 688 may be actuated to cause a vapor bubble to form within the branching microfluidic channel 691 and travel to the constriction portion 607 to apply additional shear or other forces on the cell while traveling through the constriction portion 607 and to transfect or transform the cell. Circuitry may actuate the fluidic actuator 616 to drive the flow of the cell through the microfluidic channel 618 and actuate the other fluidic actuator 688 when the cell is approaching or flowing through the constriction portion 607 to form apertures (e.g., porate) in the cell membrane of the cell.

The figures herein illustrate particular numbers of microfluidic channels, constriction portions, and fluid ejectors However, examples are not limited, and may include variety of different orientations. Although the various apparatus illustrate symmetrical designs, examples are not so limited. For example, the microfluidic devices may include high through-put and/or parallel designs.

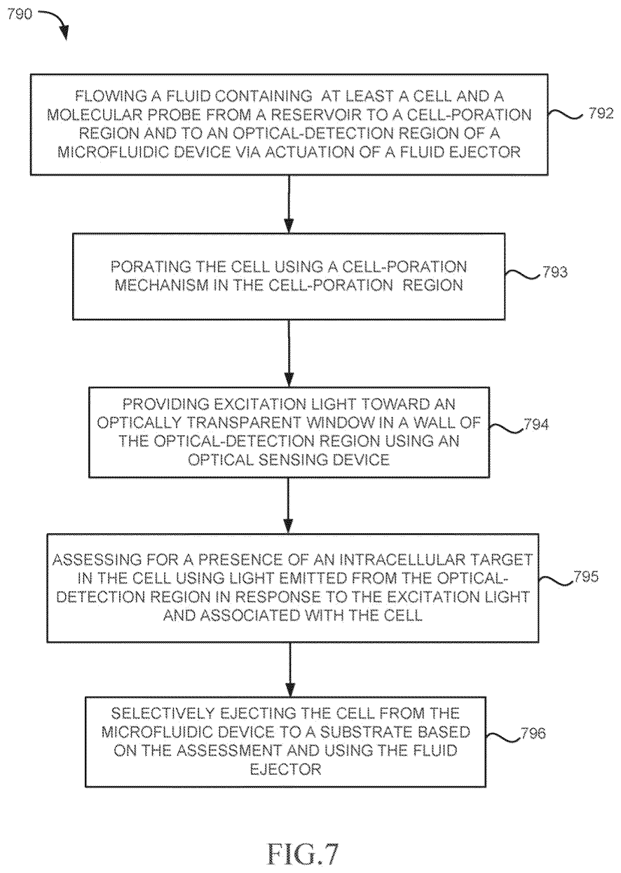

FIG. 7 illustrates an example method for porating and sorting cells using a microfluidic device, in accordance with the present disclosure. The method 790 may be performed by any of the microfluidic devices and/or apparatuses as described in connection with FIGS. 1A-6D.

At 792, the method 790 includes flowing a fluid containing at least a cell and at least a molecular probe from a reservoir to a cell-poration region and to an optical-detection region of a microfluidic device via actuation of a fluid ejector and while the cell is exposed to the molecular probe. In some examples, the fluid containing at least the cell and at least the molecular probe may be received at a fluidic inlet of the reservoir, such as via pipetting. In some examples, the fluid may include a biological sample containing a plurality of cells that is pre-mixed with the plurality of the molecular probe prior to loading into the reservoir. In some examples, the biologic sample may be loaded into the reservoir, fluid containing the plurality of molecular probes may be loaded into the reservoir or another reservoir, and the method 790 includes mixing portions of the biologic sample and the molecular probe.

At 793, the method 790 includes porating the cell using a cell-poration mechanism in the cell-poration region (and while the cell is exposed to molecular probe). In some examples, porating the cell includes forming apertures in the cell membrane of the cell by flowing the cell through the cell-poration region, wherein the cell-poration mechanism includes at least one of a constriction portion and a set of electrodes disposed with a microfluidic channel. The method further includes incubating the cell with the apertures and with the molecular probe such that the molecular probe is allowed to pass through the cell membrane and then bind to the intracellular target of the cell (that can be RNA, for example) that is present into the cytoplasm or otherwise inside the cell. For example, and as described above, porating may include at least one of mechanical porating (e.g., shearing) the cell by flowing the cell through the constriction portion, and electroporating the cell by an electric field applied across the cell via the set of electrodes of the cell-poration region in order to form the apertures in the cell membrane that the molecule probe may pass through. In some examples, porating may include electroporating. In some examples, porating may include both mechanical porating (e.g., shearing) and electroporating.

The flow of fluid is directed using fluid ejector disposed within the microfluidic channel and/or in fluidic communication with the optical-detection region. In examples that include mechanical poration or combination of mechanical and electrical poration, the cell-poration region includes a constriction portion having a first circumference that is attenuated from remaining portions of the microfluidic device. The change in circumferences between the constriction portion and remaining portions of the microfluidic device, such as a portion of the microfluidic channel associated with the cell-poration region, may impart acceleration and/or deceleration, and create compression, tensile, and shear forces on the cell. Additional forces may also occur. For example, a transection of the first circumference of the constriction portion may be smaller than a nominal diameter of the cell. In some examples, directing the flow of the cell through the constriction portion includes constricting the cell in a dimension from between ninety % to thirty % of a nominal circumference of the cell, and causing deformation of the cell and shear friction between the cell and walls of the constriction portion while flowing the cell through the constriction portion.

When the cell passes through the constriction portion and the remaining portions of the microfluidic channel, compression, tensile, and/or shear forces may be applied to the cell which cause the formation of the apertures in the cell membrane. In some examples, directing the flow of the cell through the constriction portion may include applying compression, tensile, and shear forces on the cell by directing the flow of the cell into and through the constriction portion, and removing the compression, tensile, and shear forces on the cell by directing the flow of the cell out of the constriction portion.

In examples including electroporation and/or combination of electroporation and mechanical poration, the cell-poration region includes a set of electrodes which apply an electric field to the cell as the cell travels through the cell-poration region. The electric field applied by the set of electrodes may cause the cell to form apertures in the cell membrane sufficient for the molecular probe to transition through. As previously described, the potential and electric field applied may be between about 10 KHz and about 20 mHz, between about 0.07 V/um RMS and about 0.2 V/μm RMS, and for between about 0.1 ms and about 1000 ms. The energy and/or current applied may be dependent on electrode size and conductivity of the fluid, with the conductivity of the fluid being between about 0.001 S/m and about 2 S/m.

In some examples, the method 790 further includes incubating the cell with the molecular probes to allow for the molecular probe to pass through the apertures created in the cell membrane and the apertures to close, thereby trapping the molecular probe in the cell, into the cytoplasm of the cell.

At 794, the method 790 includes providing excitation light toward an optically transparent window associated with a wall of the optical-detection region using an optical sensing device. At 795, the method 790 includes assessing for a presence of an intracellular target in the cell using light emitted from the optical-detection region in response to the excitation light and associated with the cell. For example, assessing for the presence of the intracellular target, which are in the cytoplasm of the cell, may include outputting the excitation light toward the optically transparent window by a light source of the optical sensing system, measuring the emitted light via an optical detector of optical sensing system in response to the excitation light, and detecting the presence of the intracellular target in response to the emitted light including a fluorescent signal associated with the molecular probe binding to the intracellular target.

At 796, the method 790 includes selectively ejecting the cell from the microfluidic device to a substrate based on the assessment and using the fluid ejector. In some examples, a plurality of cells are selectively ejected where selectively ejecting the plurality of cells includes, in response to detecting the presence of the intracellular target, ejecting respective cells of the plurality to select ones of a plurality of regions of the substrate, and in response to not detecting the presence of the intracellular target, ejecting respective cells of the plurality to a waste region of the plurality of regions of the substrate. For example, the substrate may include a multi-well plate, wherein at least one of the wells is a junk well.

Although the above method 790 describes flowing and processing a cell, in various examples, the fluid may contain a plurality of cells including the cell of interest and a plurality or a volume of the molecule probe. In such examples, the method 790 may include porating a plurality of cells using the cell-poration mechanism in the cell-poration region and while the plurality of cells are exposed to respective ones of the plurality of molecule probe, providing the excitation light toward the optically transparent window, assessing for the presence of the intracellular target in the plurality of cells using light emitted from the optical-detection region response to excitation light associated with the plurality of cells, and selectively ejecting the plurality of cells.

In some examples, prior to assessing the plurality of cells, the method 790 may further include detecting for the presence or not of respective ones of the plurality of cells via a sensor disposed in a microfluidic channel between the reservoir and the optical-detection region of the microfluidic device.

In some examples, the method 790 may include identifying and/or optimizing the poration parameters for a particular type of cell and/or for different types of cells by performing a set of optimization tests that include applying different poration conditions on the cell type using the microfluidic device and/or microfluidic device with a plurality of microfluidic channels having different cell-poration regions and/or constriction portions with different dimensions, different length microfluidic channels having a set of electrodes, and/or otherwise used to apply different types and/or amounts of electric fields, and measuring the resulting transfection or transformation efficiency associated with the cells. Example poration parameters include flow rate, velocity, shear force, acceleration force, deceleration force, wall friction, shear force exposure time, electric field exposure time, number of exposures to the electric field, magnitude of the electric field, frequency of the electric field, and type of buffer fluid used, among others, such as force imparted on the cell by the fluid ejector when ejecting the cell.

For example, the amount of shear force may be adjusted by changing the flow rate in a particular microfluidic channel for a plurality of cells of a particular cell type, and/or by using multiple microfluidic channels having constriction portions with different dimensions and/or by changing the flow rate in the microfluidic channel or multiple microfluidic channels. As other examples, the magnitude and/or frequency of electric field applied to the cell may be adjusted by changing the flow rate in a particular microfluidic channel for a plurality of cells of a particular cell type, by using multiple microfluidic channels having different lengths for the cell-poration region and/or different numbers of exposures to the electric field (e.g., the set of electrodes cross the channel multiple times or includes multiple sets of electrodes), and/or by changing the flow rate in the multiple microfluidic channels. The flow rate may be changed, for example, by adjusting the velocity of the flow using the fluid ejector, such as by activating a resistor with different pulse widths, pulse frequencies, and/or for a different amount of time, and/or activating different amounts of resistors to impart different amounts of momentum to the fluid and thereby change the fluid velocity. The flow rate, which may be referred to as a volume flow rate, may be defined as:

$$Flow rate = velocity \times cross - sectional area of the microfluidic channel ,$$

where the cross-sectional area is defined by the circumference and/or diameter of the microfluidic channel. The flow rate and amount of constriction may impact the compression, tensile, and/or shear forces applied on the cell. For example, by adjusting the actuation of the fluid ejector and/or using a cell-poration region with different dimensions (e.g., different lengths, different number of turns or electrodes, an adjusted circumference for the constriction portion), the flow rate may change and which causes different amounts of electric fields and/or shear force to be applied on respective cells, with the adjusted circumference further causing a different compression force applied on respective cells. In some examples, a change in the length of the cell-poration region may cause application of the electric field and/or shear force for a different amount of time. In some examples, the length, shape, and taper of the circumference transitions between constriction portions and remaining portions of the microfluidic device may be used to adjust the flow rate changes and the associated amount of compression, tensile, and/or shear forces on the cell membrane which may impact the formation and size of the apertures. For example, the taper of the circumference transitions between respective constriction portions and respective remaining portions of the microfluidic device may contribute and/or cause compression, tensile, and/or shear forces due to acceleration and/or deceleration forces applied as the respective cells enter and exit the constriction portions. A plurality of cells may be tested by exposing the cells to the different amounts of electric fields and/or different compression, tensile, and/or shear forces using the apparatus, and analyzing the resulting transfection or transformation efficiency of the cells. For example, cells of different sizes and/or pores of different diameters may be formed by adjusting an exposure time to the electric field, a magnitude of the electric field, a frequency of the electric field, buffer conductivity, and/or buffer composition. In some examples, the exposure time may be adjusted by adjusting a flow rate, adjusting the length of the cell-poration region, and/or adjusting the time the electrodes are actuated. The magnitude and/or frequency of the electric field may be adjusted by the type or size of electrodes used and/or by selective actuation of the electrodes. The process may be repeated for a plurality of different cell types.

In some examples, a list of set electric fields and/or shear forces for the different types of cells may be generated from the optimization test and stored by the apparatus, such as by circuitry 124 of FIG. 1B. For example, circuitry coupled to the fluid ejector may store the list, which identifies the set electric fields and/or shear forces for the different types of cells and/or the set flow rate used to cause the set electric field and/or shear force. In some examples in which the microfluidic device includes a plurality of microfluidic channels, the list may identify different flow rates for different constriction portions which may be used to apply the set shear force. In some examples, the apparatus may include the stored list, and may further optimize the set shear forces over time using feedback data.

Circuitry, such as circuitry 124 and 624, included a processor, machine readable instructions, and other electronics for communicating with and controlling the fluidic pumps, and other components of the apparatus, such as the sensor, the fluidic pump(s) and/or resistor(s), and other components. The circuitry may receive data from a host system, such as a computer, and includes memory for temporarily storing data. The data may be sent to the apparatus along an electronic, infrared, optical, or other information transfer path. A processor may be a CPU, a semiconductor-based microprocessor, a GPU, a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one IC, other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, RAM, an EEPROM, a storage device, an optical disc, etc. In some examples, the computer-readable storage medium may be a non-transitory storage medium, where the term ‘non-transitory’ does not encompass transitory propagating signals.

A fluid may include a biologic sample in a fluid. A biological sample, as used herein, refers to any biological material, collected from a subject. Examples of biologic samples include, but are not limited to, whole blood, blood plasma, and other body fluids, as well as tissue cell cultures obtained from humans, plants, or animals. Such biologic samples may contain any viral or cellular material, including all prokaryotic or eukaryotic cells, viruses, bacteriophages, mycoplasmas, protoplasts, and organelles. The biological material may comprise all types of mammalian and non-mammalian animal cells, plant cells, algae including blue-green algae, fungi, bacteria, protozoa, etc. Non-limiting sample examples include whole blood and blood-derived products such as plasma, serum and buffy coat, urine, feces, cerebrospinal fluid or other body fluids, tissues, cell cultures, cell suspensions, etc. The term “fluid”, as used herein, refers to any substance that flows under applied forces. In some examples, the fluid includes the biologic sample including an analyte and/or a reagent or reactant, among others.

The various ranges provided herein include the stated range and any value or sub-range within the stated range. Furthermore, when “about” is utilized to describe a value or percentage this includes, refers to, and/or encompasses variations (up to +/−ten %) from the stated value or percentage. In describing and claiming the examples disclosed herein, the singular forms “a”, “an”, and “the” include plural referents unless the context clearly dictates otherwise.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A microfluidic device, comprising:
a reservoir to contain a fluid including at least a cell and at least a molecular probe;
a cell-poration region fluidically coupled to the reservoir and including a cell-poration mechanism;
an optical-detection region fluidically coupled to the cell-poration region and including an optically transparent window associated with a wall of the optical-detection region; and
a fluid ejector fluidically coupled to the optical-detection region.

2. The device of claim 1, wherein the fluid ejector includes an ejection nozzle and a fluidic actuator disposed with an ejection chamber fluidically coupled to the optical-detection region.

3. The device of claim 1, wherein the cell-poration mechanism includes a set of electrodes.

4. The device of claim 3, wherein the cell-poration region includes a microfluidic channel that is serpentine-shaped and with the set of electrodes extending therethrough at a plurality of portions of the microfluidic channel.

5. The device of claim 1, wherein the cell-poration mechanism includes a constriction portion.

6. The device of claim 5, wherein the cell-poration region includes a microfluidic channel with the constriction portion that includes a circumference that is attenuated from remaining portions of the microfluidic channel.

7. The device of claim 5, wherein the cell-poration mechanism further includes a set of electrodes including a first electrode disposed upstream of the constriction portion and a second electrode disposed downstream of the constriction portion.

8. The device of claim 1, further including a first sensor region disposed between the cell-poration region and the optical-detection region, the first sensor region including a first microfluidic channel with a first sensor disposed with the first microfluidic channel.

9. The device of claim 8, further including a second sensor region disposed between the reservoir and the cell-poration region, the second sensor region including a second microfluidic channel with a second sensor disposed with the second microfluidic channel.

10. An apparatus, comprising:
a microfluidic device including:
a reservoir to store a fluid containing at least a cell and at least a molecular probe;
a cell-poration region fluidically coupled to the reservoir and including a set of electrodes;
an optical-detection region fluidically coupled to the cell-poration region and including an optically transparent window associated with a wall of the optical-detection region; and
a fluid ejector fluidically coupled to the optical-detection region;
an optical sensing device to measure emitted light responsive to excitation light provided toward the optical-detection region; and
circuitry communicatively coupled to the microfluidic device and the optical sensing device to drive flow of the fluid through the microfluidic device via the fluid ejector and to detect binding of the molecular probe to an intracellular target in the cell via the measured emitted light.

11. The apparatus of claim 10, further including a substrate including a plurality of regions and a stage coupled to the substrate, wherein:
the circuitry is communicatively coupled to the stage to instruct the stage to move the substrate relative to the fluid ejector, such that the fluid ejector is aligned with a select region of the plurality of regions of the substrate; and
the fluid ejector is to selectively eject a fluid droplet of the fluid containing the cell from the microfluidic device to the select region of the plurality of regions of the substrate.

12. The apparatus of claim 11, wherein the fluid includes a plurality of cells including the cell of interest and a plurality of the molecular probe, and the circuitry is to:
assess for a presence of the intracellular target in the plurality of cells via the measured emitted light associated with each of the plurality of cells; and
actuate the fluid ejector to cause selective ejection of the plurality of cells from the microfluidic device to a plurality of regions of the substrate and based on the assessed presence.

13. The apparatus of claim 10, wherein the cell-poration region further includes a microfluidic channel with a constriction portion, wherein a first electrode of the set of electrodes is disposed upstream of the constriction portion and a second electrode of the set of electrodes is disposed downstream of the constriction portion.

14. A method, comprising:
flowing a fluid containing at least a cell and at least a molecular probe from a reservoir to a cell-poration region and to an optical-detection region of a microfluidic device via actuation of a fluid ejector and while the cell is exposed to the molecular probe;
porating the cell using a cell-poration mechanism in the cell-poration region;
providing excitation light toward an optically transparent window associated with a wall of the optical-detection region using an optical sensing device;
assessing for a presence of an intracellular target in the cell using light emitted from the optical-detection region in response to the excitation light and associated with the cell; and
selectively ejecting the cell from the microfluidic device to a substrate based on the assessment and using the fluid ejector.

15. The method of claim 14, wherein porating the cell includes:
forming apertures in the cell membrane of the cell by flowing the cell through the cell-poration region, wherein the cell-poration mechanism includes at least one of a constriction portion and a set of electrodes disposed with a microfluidic channel, wherein porating the cell includes at least one of:
mechanical porating the cell by flowing the cell through the constriction portion; and
electroporating the cell by an electric field applied across the cell via the set of electrodes of the cell-poration region; and
the method further including incubating the cell with the apertures with the molecular probe such that the molecular probe is allowed to pass through one of the apertures and bind to the intracellular target of the cell.
